SEQUENCE LISTING (6 sequences)

SEQ ID NO: 1 - DNA, 3306 bp, Homo sapiens
  CDS (47)..(2791)
gattgaaaggcggttgtggtgcaaaggaaaacccacaggccaaggaatgggaagaccaaggttgacacttgtttgtcaagtgtcaataatcatctctgcccgggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacttttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatcggcctccaacacaaccgcatctgggaaattggagctgacaccttcagccagctgagctccctgcaagccctggatcttagctggaacgccatccggtccatccaccccgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgtccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcgctgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagctttgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtccctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttggaagagattacaaaaaaaaaaaaaaaaaaaaaaaaa

SEQ ID NO: 2 - protein, 915 aa, Homo sapiens
MGRPRLTLVCQVSIIISARDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHFYDNPIQFVGRSAFQYLPKLHTLSLNGAMDIQEFPDLKGTTSLEILTLTRAGIRLLPSGMCQQLPRLRVLELSHNQIEELPSLHRCQKLEEIGLQHNRIWEIGADTFSQLSSLQALDLSWNAIRSIHPEAFSTLHSLVKLDLTDNQLTTLPLAGLGGLMHLKLKGNLALSQAFSKDSFPKLRILEVPYAYQCCPYGMCASFFKASGQWEAEDLHLDDEESSKRPLGLLARQAENHYDQDLDELQLEMEDSKPHPSVQCSPTPGPFKPCEYLFESWGIRLAVWAIVLLSVLCNGLVLLTVFAGGPVPLPPVKFVVGAIAGANTLTGISCGLLASVDALTFGQFSEYGARWETGLGCRATGFLAVLGSEASVLLLTLAAVQCSVSVSCVRAYGKSPSLGSVRAGVLGCLALAGLAAALPLASVGEYGASPLCLPYAPPEGQPAALGFTVALVMMNSFCFLVVAGAYIKLYCDLPRGDFEAVWDCAMVRHVAWLIFADGLLYCPVAFLSFASMLGLFPVTPEAVKSVLLVVLPLPACLNPLLYLLFNPHFRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLAFASHV

SEQ ID NO: 3 - protein, 915 aa, Homo sapiens; identical to SEQ ID NO: 2
  mat_peptide (20)..()

SEQ ID NO: 4 - DNA, 3451 bp, Homo sapiens
  CDS (47)..(1090)
gattgaaaggcggttgtggtgcaaaggaaaacccacaggccaaggaatgggaagaccaaggttgacacttgtttgtcaagtgtcaataatcatctctgcccgggacctcagcatgaacaacctcacagagcttcagcctggcctcttccaccacctgcgcttcttggaggagctgcgtctctctgggaaccatctctcacacatcccaggacaagcattctctggtctctacagcctgaaaatcctgatgctgcagaacaatcagctgggaggaatccccgcagaggcgctgtgggagctgccgagcctgcagtcgctgcgcctagatgccaacctcatctccctggtcccggagaggagctttgaggggctgtcctccctccgccacctctggctggacgacaatgcactcacggagatccctgtcagggccctcaacaacctccctgccctgcaggccatgaccctggccctcaaccgcatcagccacatccccgactacgcgttccagaatctcaccagccttgtggtgctgcatttgcataacaaccgcatccagcatctggggacccacagcttcgaggggctgcacaatctggagacactagacctgaattataacaagctgcaggagttccctgtggccatccggaccctgggcagactgcaggaactggggttccataacaacaacatcaaggccatcccagaaaaggccttcatggggaaccctctgctacagacgatacacctttatgataacccaatccagtttgtgggaagatcggcattccagtacctgcctaaactccacacactatctctgaatggtgccatggacatccaggagtttccagatctcaaaggcaccaccagcctggagatcctgaccctgacccgcgcaggcatccggctgctcccatcggggatgtgccaacagctgcccaggctccgagtcctggaactgtctcacaatcaaattgaggagctgcccagcctgcacaggtgtcagaaattggaggaaatgagaagtggggggctctactttcttcctcccagtgctcggggggcatggagcggagccagggtctgagcctgccggctcatccagcctctcttgctgccctagcggcctccaacacaaccgcatctgggaaattggagctgacaccttcagccagctgagctccctgcaagccctggatcttagctggaacgccatccggtccatccaccctgaggccttctccaccctgcactccctggtcaagctggacctgacagacaaccagctgaccacactgcccctggctggacttgggggcttgatgcatctgaagctcaaagggaaccttgctctctcccaggccttctccaaggacagtttcccaaaactgaggatcctggaggtgccttatgcctaccagtgctgtccctatgggatgtgtgccagcttcttcaaggcctctgggcagtgggaggctgaagaccttcaccttgatgatgaggagtcttcaaaaaggcccctgggcctccttgccagacaagcagagaaccactatgaccaggacctggatgagctccagctggagatggaggactcaaagccacaccccagtgtccagtgtagccctactccaggccccttcaagccctgtgagtacctctttgaaagctggggcatccgcctggccgtgtgggccatcgtgttgctctccgtgctctgcaatggactggtgctgctgaccgtgttcgctggcgggcctgcccccctgcccccggtcaagtttgtggtaggtgcgattgcaggcgccaacaccttgactggcatttcctgtggccttctagcctcagtcgatgccctgacctttggtcagttctctgagtacggagcccgctgggagacggggctaggctgccgggccactggcttcctggcagtacttgggtcggaggcatcggtgctgctgctcactctggccgcagtgcagtgcagcgtctccgtctcctgtgtccgggcctatgggaagtccccctccctgggcagcgttcgagcaggggtcctaggctgcctggcactggcagggctggccgccgcactgcccctggcctcagtgggagaatacggggcctccccactctgcctgccctacgcgccacctgagggtcagccagcagccctgggcttcaccgtggccctggtgatgatgaactccttctgtttcctggtcgtggccggtgcctacatcaaactgtactgtgacctgccgcggggcgactttgaggccgtgtgggactgcgccatggtgaggcacgtggcctggctcatcttcgcagacgggctcctctactgtcccgtggccttcctcagcttcgcctccatgctgggcctcttccctgtcacgcccgaggccgtcaagtctgtcctgctggtggtgctgcccctgcctgcctgcctcaacccactgctgtacctgctcttcaacccccacttccgggatgaccttcggcggcttcggccccgcgcaggggactcagggcccctagcctatgctgcggccggggagctggagaagagctcctgtgattctacccaggccctggtagccttctctgatgtggatctcattctggaagcttctgaagctgggcggccccctgggctggagacctatggcttcccctcagtgaccctcatctcctgtcagcagccaggggcccccaggctggagggcagccattgtgtagagccagaggggaaccactttgggaacccccaaccctccatggatggagaactgctgctgagggcagagggatctacgccagcaggtggaggcttgtcagggggtggcggctttcagccctctggcttggcctttgcttcacacgtgtaaatatccctccccattcttctcttcccctctcttccctttcctctctccccctcggtgaatgatggctgcttctaaaacaaatacaaccaaaactcagcagtgtgatctatagcaggatggcccagtacctggctccactgatcacctctctcctgtgaccatcaccaacgggtgcctcttggcctggctttcccttggccttcctcagcttcaccttgatactgggcctcttccttgtcatgtctgaagctgtggaccagagacctggacttttgtctgcttaagggaaatgagggaagtaaagacagtgaaggggtggagggttgatcagggcacagtggacagggagacctcacagagaaaggcctggaaggtgatttcccgtgtgactcatggataggatacaaaatgtgttccatgtaccattaatcttgacatatgccatgcataaagacttcctattaaaataagctttgggagagaaaaaaaaaaaaaaaaaaaaaaaaaaaaaaaaaaaaaaaaaaaaaaaaaaaaaaaaaaaaaaaaaaaaaaaa

SEQ ID NO: 5 - protein, 348 aa, Homo sapiens
MGRPRLTLVCQVSIIISARDLSMNNLTELQPGLFHHLRFLEELRLSGNHLSHIPGQAFSGLYSLKILMLQNNQLGGIPAEALWELPSLQSLRLDANLISLVPERSFEGLSSLRHLWLDDNALTEIPVRALNNLPALQAMTLALNRISHIPDYAFQNLTSLVVLHLHNNRIQHLGTHSFEGLHNLETLDLNYNKLQEFPVAIRTLGRLQELGFHNNNIKAIPEKAFMGNPLLQTIHLYDNPIQFVGRSAFQYLPKLHTLSLNGAMDIQEFPDLKGTTSLEILTLTRAGIRLLPSGMCQQLPRLRVLELSHNQIEELPSLHRCQKLEEMRSGGLYFLPPSARGAWSGARV

SEQ ID NO: 6 - protein, 348 aa, Homo sapiens; identical to SEQ ID NO: 5
  mat_peptide (20)..()

(19) United States
(12) Patent Application Publication
(10) Pub. No.: US 2006/0205924 A1
(43) Pub. Date: Sep. 14, 2006

(54) Spliced variants of lgr6

(76) Inventor: Marco Muda, Brookline, MA (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 10/540,808

(22) PCT Filed: Dec. 23, 2003
(86) PCT No.: PCT/EP03/51091

§ 371 Date: Apr. 14, 2006

Related U.S. Application Data
(60) Provisional application No. 60/436,001, filed on Dec. 26, 2002.

(51) Int. Cl. C07K 14/72; C07H 21/04; C12P 21/06; A61K 48/00; A61K 39/395; C07K 16/28

(52) U.S. Cl. 530/350; 530/388.22; 424/93.21; 424/143.1; 435/69.1; 435/320.1; 435/325; 536/23.5

(57) ABSTRACT

The present invention provides Leucine-Rich Repeat-Containing G-Protein Coupled Receptor-6 (LGR6-Vs) polypeptides and nucleic acid molecules encoding the same. The invention also provides selective binding agents, vectors, host cells, and methods for producing LGR6-SVs polypeptides. The invention further provides pharmaceutical compositions and methods for the diagnosis, treatment, amelioration, and/or prevention of diseases, disorders, and conditions associated with LGR6-SVs polypeptides.

### FIELD OF THE INVENTION

The present invention relates to Leucine-Rich Repeat-Containing G-Protein Coupled Receptor-6 (LGR-6-SVs) polypeptides and nucleic add molecules encoding the same. The invention also relates to selective binding agents, vectors, host cells, and methods for producing LGR-6-SVs polypeptides. The invention further relates pharmaceutical compositions and methods for the diagnosis, treatment, amelioration, and/or prevention of diseases, disorders, and conditions associated with LGR-6-SVs polypeptides.

### BACKGROUND OF THE INVENTION

G-protein coupled receptors (GPCRs) are seven-transmembrane-domain proteins that mediate signal transduction of a diverse number of ligands through heterotrimeric G proteins (Strader, C. D. et al. (1994) Annu. Rev. Biochem. 63: 101-132), G protein-coupled receptors (GPCRs), along with G-proteins and effector proteins (e.g., intracellular enzymes and channels), are the components of a modular signaling system.

Upon ligand binding to an extracellular portion of a GPCR, different G proteins are activated, which in turn modulate the activity of different intracellular effector enzymes and ion channels (Gutkind, J. S. (1998) J Biol. Chem. 273: 1839-1842; Selbie, L. A. and Hill, S. J. (1998) Trends Pharmacol. Sci. 19:87-93).

Members of a glycoprotein hormone receptor subfamily of G-protein coupled receptor (GPCR)/seven-transmembrane domain receptors are characterized by a relatively large (more than 330 amino acids) ligand-binding N-terminal extracellular domain that contains a unique leucine-rich repeat structure (Dufau, 1998, Annu. Rev. Physiol. 60:461-96). Among the members of this subfamily are the thyroid stimulating hormone (TSH) receptor, the follicle stimulating hormone (FSH) receptor, and the luteinizing hormone (LH)/chorionic gonadotropin (CG) receptor.

Recently, a number of orphan GPCRs having significant homology to the glycoprotein hormone receptor subfamily have been described. These novel members include the leucine rich repeat-containing G-protein coupled receptor (LGR) 4 (Hsu et al., 1998, Mol. Endocrinol. 12:1830-45; PCT Publication No. WO 99115545), LGR5 (McDonald et al., 1998, Biochem. Biophys. Res. Commun. 247:266-70; Hsu et al., 1998, Mol. Endocrinol. 12:1830-45; PCT Publication No. WO 99/15660), LGR6 (European Patent App. No. EP 0 950 711 A2), LGR7 (PCT Publication No. WO 99/48921; Hsu et al., 2000, Mol. Endocrinol. 14:1257-71) and LGR8 (Hsu eta., 2002, Science 295: 671-4).

The N-terminal extracellular domain of the glycoprotein hormone receptor subfamily retains ligand-binding capability in the absence of both the transmembrane domains and the cytoplasmic C-terminal region. In fact, the N-terminal extracellular domains of the LH, FSH, and TSH receptors, when expressed recombinantly, have been shown to selectively neutralize LH, FSH, or TSH-induced signal transduction (Osuga et al., 1997, Mol. Endocrinol. 11:1659-68). In other words, the soluble extracellular domain has been shown to act as a functional antagonist of the signaling pathway of the receptor from which the extracellular domain was derived.

Because hormones and receptors play a prominent role in a variety of physiological processes, there is continued interest in the identification of novel receptors and their ligands, as well as the genes encoding the same.

GPCRs are of critical importance to several systems including the endocrine system, the central nervous system and peripheral physiological processes.

Evolutionary analysis suggests that the ancestor of these proteins originally developed in concert with complex body plans and nervous systems. The GPCR genes and gene-products are believed to be potential causative agents of disease (Spiegel et al. (1993) J. Clin. Invest. 92:1119-1125); McKusick and Amberger (1993) J. Med. Genet 30:1-26).

For example, specific defects in the rodopsin gene and the V2 vasopressin receptor gene have been shown to cause various forms of autosomal dominant and autosomal recessive retinitis pigmentosa (see Nathans et al. (1992) Annual Rev. Genet 26:403-424), and nephrogenic diabetes insipidus (Holtzman et al. (1993) Hum. Mol. Genet. 2:1201-1204).

Given the important biological roles and properties of GPCRs, there exists a need for the identification of novel genes encoding such proteins as well as for the discovery of modulators of such molecules for use in regulating a variety of normal and/or pathological cellular processes.

Relevant Literature References of interest include: El Tayar, N, “Advances in the Molecular Understanding of Gonadotropins-Receptors Interactions,” Mol. Cell. Endocrinol. (Dec. 20, 1996), 125: 65-70; Bhowmick et al., “Determination of Residues Important in Hormone Binding to the Extracellular Domain of the Luteinizing Hormone/Chorionic Gonadotropin Receptor by Site-Directed Mutagenesis and Modeling,” Mol. Endocrinol. (September 1996) 10: 1147-1159; Thomas et al., “Mutational Analyses of the Extracellular Domain of the Full-Length Lutropin/Choriogonadotropin Receptor Suggest Leucine-Rich Repeats 1-6 are Involved in Hormone Binding,” Mol. Endocrinol. (June 1996) 10:760-768; Segaloff & Ascoli, “The Gonadotropin Receptors: Insights from the I Cloning of their cDNAs,” Oxf. Rev. Reprod. Biol. (1992) 14: 141-168; Braun et al., “Amino-Terminal Leucine-Rich Repeats in Gonadotropin Receptors Determine Hormone Selectivity,” EMBO J (July 1991) 10: 1885-1890; and Segaloff et al., “Structure of the Lutropin/Choriogonadotropin Receptor,” Recent Prog. Horm. Res. (1990) 46: 261-301.

### SUMMARY OF THE INVENTION

The present invention relates to two novel and distinct LGR6 alternative splicing variants, specifically LGR6.1 and LGR6.2 (hereafter collectively indicated as “LGR6-SVs”). The LGR6 coding sequence consists of at least 18 exons that encode a large N-terminal leucine-rich repeat-containing extracellular domain, seven predicted transmembrane domains, and a cytoplasmic C-terminal region. The LGR6.1 coding sequence is identical to the LGR6 coding sequence with the exception that the LGR6.1 coding sequence comprises one novel exon encoding a novel leader peptide and two novel internal exons. The LGR6.2 coding sequence consists of the same exons present in LGR6.1 plus an extended exon that introduces a stop codon resulting in a truncation of LGR6.1 coding sequence, lacking the transmembrane domains and the cytoplasmic C-terminal region. Thus, LGR6.2 is a secreted, N-terminal extracellular domain version of LGR6.1 and likely functions as an antagonist of the LGR6.1 signaling pathway. LGR6.2 is truncated near the C-terminal end of the N-terminal extracellular domain by virtue of the alternative use of splicing acceptor site that results in the introduction of additional 22 amino acids and a stop codon.

The invention provides for an isolated nucleic acid molecule comprising a nucleotide sequence selected from the group consisting of.

(a) the nucleotide sequence as set forth in any of SEQ ID NO: 1 and SEQ ID NO: 3;

(b) a nucleotide sequence encoding the polypeptide as set forth in any of SEQ ID NO: 2, and SEQ ID NO: 4;

(c) a nucleotide sequence which hybridize under stringent conditions with one of

(a) or (b); or

the nucleotide sequence 1-102 of SEQ ID NO: 1 or SEQ ID NO:3; or

the nucleotide sequence 319-606 of SEQ ID NO:1 or SEQ ID NO: 3; or

the nucleotide sequence 1027-1201 of SEQ ID NO: 3.

The present invention also provides for an expression vector comprising the isolated nucleic acid molecules as set forth herein, recombinant host cells comprising the recombinant nucleic acid molecules as set forth herein, and a method of producing an LGR6-SVs polypeptide comprising culturing the host cells and optionally isolating the polypeptide so produced.

A transgenic non-human animal comprising a nucleic acid molecule encoding an LGR6-SVs polypeptide is also encompassed by the invention. The LGR6 SVS nucleic acid molecules are introduced into the animal in a manner that allows expression and increased levels of an LGR6-SVs polypeptide, which may include increased circulating levels. Alternatively, the LGR6-SVs nucleic acid molecules are introduced into the animal in a manner that prevents expression of endogenous LGR6-SVs polypeptide (i.e., generates a transgenic animal possessing an LGR6-SVs polypeptide gene (knock-out).

The transgenic non-human animal is preferably a mammal, and more preferably a rodent, such as a rat or a mouse.

Also provided are derivatives of the LGR6-SVs polypeptides of the present invention, which includes polypeptide comprising the amino acid sequence of SEQ ID NO: 2 or SEQ ID NO: 4.

Additionally provided are selective binding agents such as antibodies and peptides capable of specifically binding the LGR6-SVs polypeptides of the invention. Such antibodies and peptides may be agonistic or antagonistic.

Pharmaceutical compositions comprising the nucleotides, polypeptides, or selective binding agents of the invention and one or more pharmaceutically acceptable formulation agents are also encompassed by the invention. The pharmaceutical compositions are used to provide therapeutically effective amounts of the nucleotides or polypeptides of the present invention. The invention is also directed to methods of using the polypeptides, nucleic acid molecules, and selective binding agents.

The LGR6-SVs polypeptides and nucleic acid molecules of the present invention may be used to treat, prevent, ameliorate, and/or detect diseases and disorders, including those recited herein.

The present invention also provides a method of assaying test molecules to identify a test molecule that binds to an LGR6-SVs polypeptide. The method comprises contacting an LGR6-SV polypeptide with a test molecule to determine the extent of binding of the test molecule to the polypeptide. The method further comprises determining whether such test molecules are agonists or antagonists of an LGR6-SVs polypeptide. The present invention further provides a method of testing the impact of molecules on the expression of LGR6-SVs polypeptide or on the activity of LGR6-SVs polypeptide.

Methods of regulating expression and modulating (i.e., increasing or decreasing) levels of an LGR6-SVs polypeptide are also encompassed by the invention. One method comprises administering to an animal a nucleic acid molecule encoding an LGR6-SVs polypeptide. In another method, a nucleic acid molecule comprising elements that regulate or modulate the expression of an LGR6-SVs polypeptide may be administered. Examples of these methods include gene therapy, cell therapy, and anti-sense therapy as further described herein.

LGR6-SVs polypeptides can be used for identifying ligands thereof. Various forms of “expression cloning have been used for cloning ligands for receptors (See, e.g., Io Davis et al., 1996, Cell, 87:1161-69). These and other LGR6-SVs ligand cloning experiments are described in greater detail herein. Isolation of the LGR6-SVs ligand(s) allows for the identification or development of novel agonists and/or antagonists of the LGR6-SVs signaling pathway. Such agonists and antagonists include LGR6-SVs ligand(s), anti-LGR6-SVs ligand antibodies and derivatives thereof, small molecules, or antisense oligonucleotides, any of which can be used for potentially treating one or more diseases or disorders, including those recited herein.

### BRIEF DESCRIPTION OF THE FIGURE

FIG. 1 illustrates nucleotide sequence alignment of LGR6.1, LGR6.2 and LGR6 using the ALIGNX application of Vector NTI Suite (Informax, Inc, Bethesda).

FIG. 2 reports the tissue expression distribution of the 2 novel LGR6-SVs.

FIG. 3 illustrates the amino acid alignment of LGR6, LGR6.1 and LGR6.2 using the ClustalW alignment tool. In addition, the YAYQCC and GPFKPCEY hinge-conserved sequences as well as the RGD tripeptide sequence are indicated.

FIG. 4 reports the SMART Domains alignment of LGR6, LGR6.1 and LGR6.2.

### DETAILED DESCRIPTION OF THE INVENTION

The section headings used herein are for organizational purposes only and are not to be construed as limiting the subject matter described. All references cited in this application are expressly incorporated by reference herein.

It will be appreciated that LGR6.1 is membrane-bound polypeptides having an N-terminal extracellular domain, multiple transmembrane domains, and a C-terminal cytoplasmic domain. Accordingly, LGR6.1 can be useful as target for agonistic or antagonistic molecules, including, but not limited to, antibodies, fusion polypeptides, carbohydrates, polynucleotides (such as antisense oligonucleotides), or small molecular weight organic molecules.

Additionally, it will be understood that the N-terminal extracellular domains of LGR6.1 and LGR6.2 can be used as antagonists of the LGR6 signaling pathway, for example, where the N-terminal extracellular domain is fused to an Fc portion of an antibody.

It will also be appreciated that LGR6.2 is a secreted form of the N-terminal extracellular domain of LGR6.1. In this regard LGR6.2 may act as an antagonist of the LGR6 ligand(s). LGR6.2 can also be used as a target for antagonistic and agonistic molecules, including, but not limited to, antibodies, fusion polypeptides, carbohydrates, polynucleotides (such as antisense oligonucleotides), or small molecular weight organic molecules. For example, an antagonist specific for LGR6.2 would inhibit the antagonistic activity of LGR6.2, thus enhancing the activity of LGR6 ligand(s) and/or enhancing signaling through LGR6 receptors. Conversely an agonist specific for LGR6.1 would enhance the antagonistic activity of LGR6.2, thus diminishing the activity of LGR6 ligand(s) and/or diminishing signaling through LGR6 receptors.

Definitions

The terms “LGR6-SVs gene” or “LGR6-SVs nucleic acid molecule” or “LGR6-SVs polynucleotide” refer to an isolated nucleic acid molecule comprising a nucleotide sequence selected from the group consisting of.
(a) the nucleotide sequence as set forth in any of SEQ ID NO: 1 and SEQ ID NO: 3;
(b) a nucleotide sequence encoding the polypeptide as set forth in any of SEQ ID NO: 2, SEQ ID NO: 4;
(c) a nucleotide sequence which hybridizes under moderately stringent conditions with one of:
   (a) or (b); or
   the nucleotide sequence 1-102 of SEQ ID NO: 1 or SEQ ID NO:3; or
   the nucleotide sequence 319-606 of SEQ ID NO:1 or SEQ ID NO: 3; or the nucleotide sequence 1027-1201 of SEQ ID NO: 3.

The term “isolated nucleic acid molecule” refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use.

The term “nucleic acid sequence” or “nucleic acid molecule” refers to a DNA or RNA sequence. The term encompasses molecules formed from any of the known base analogs of DNA and RNA such as, but not limited to 4-acetylcytosine, 8-hydroxy-N-6-methyladenosine, aziridinyl-cytosine, pseudoisocytosine, 5 (carboxyhydroxylmethyl) uracil, 5-fluorouracil, 5-bromouracil, 5 carboxymethylaminomethyl-2-thiouracil, 5-carboxymethylaminomethyluracil, dihydrouracil, inosine, N6-iso-pentenyladenine, 1-methyladenine, 1-methylpseudouracil, 1-methylguanine, 1-methylinosine, 2,2-dimethylguanine, 2 methyladenine, 2-methylguanine, 3-methylcytosine, 5-methylcytosine, N6 methyladenine, 7-methylguanine, 5-methylaminomethyluracil, 5-methoxyamino methyl-2-thiouracil, beta-D-mannosylqueosine, 5′-methoxycarbonyl-methyluracil, 5 methoxyuracil, 2-methylthio-N6-isopentenyladenine, uracil-5-oxyacetic acid methylester, uracil-oxyacetic acid, oxybutoxosine, pseudouracil, queosine, 2 thiocytosine, 5-methyl-2-thiouracil, 2-thiouracil, 4-thiouracil, 5-methyluracil, N uracil-oxyacetic acid methylester, uracil-5-oxyacetic acid, pseudouracil, queosine, 2-thiocytosine, and 2,6-diaminopurine.

The present invention also refers to recombinant DNA molecules, which hybridize with the DNA sequence coding for LGR6-SVs or fragments thereof. The gene can contain, or not, the natural introns and can be obtained for example by extraction from appropriate cells and purification with known methods.

Appropriate preparations of DNA, as human genomic DNA, are cut in the appropriate way, preferably with restriction enzymes, and the so obtained fragments are introduced in appropriate recombinant vectors in order to form a DNA library. Such vectors can be selected with synthetic oligonucleotide probes in order to identify a sequence encoding the LGR6-SVs according to the invention.

On the other hand, the corresponding mRNA can be isolated from the cells expressing the LGR6-SVs and used to produce the complementary DNA (cDNA) with known methods. This cDNA after having been converted in the double helix, can be introduced in an appropriate vector which can afterwards be used for transforming an appropriated host cell.

The resulting cultures are then selected with an appropriate probe in order to obtain the cDNA encoding the targeted sequences.

Once the wanted done is isolated, the cDNA can be manipulated essentially in the same way as the genomic DNA. The cDNA does not contain introns.

Because of the degeneration of the genetic code, various codons can be used for encoding a specific amino acid, so that one or more oligonucleotides can be produced, each of them being able to encode fragments of LGR6-SVs. However only one member of this pool possesses the nucleotide sequence identical to that of the gene. Its presence in the pool and its capacity of hybridizing with the DNA also in the presence of other members of the pool makes it possible the use of the group of non fractioned oligonucleotides in the same way as a single oligonucleotide could be used for cloning the gene encoding the targeted peptide.

Alternatively, a single oligonucleotide containing the sequence which is theoretically the most probable being able of encoding the genic fragments of LGR6-SVs (according to what described in the “rules for the use of codons” In Lathe R, et al. J. Molec. Biol. 183:1-12 (1985)) allows the identification the complementary DNA encoding LGR6-SVs or a fragment thereof.

The processes for hybridizing the nucleic acids are known and described, for example in Maniatis T. et al. Molecular Cloning: A laboratory manual, Cold Spring Harbor Press, Cold Spring Harbor, N.Y., 1982) and in Haymes B. T. et al. Nucleic Acid Hybridization: A practical approach, IRL Press, Oxford, England, (1985). Through the hybridization using said probe or group of nucleotide probes it is possible to identify in a genomic or cDNA gene library the DNA sequences capable of such hybridization which are thereafter analyzed to confirm that they encode the polypeptide according to the invention. The oligonucleotide, which contains such complementary sequence can be synthesized and used as probe to identify and isolate the gene of the polypeptide according to the invention (Maniatis T. et al. ibid.). Once the appropriate oligonucleotide specific for the LGR6-SVs is selected using the above said method, it is possible to synthesize and hybridize it with a DNA, or preferably with a cDNA derived from cells capable of expressing the wanted gene preferably after the source of cDNA was enriched of wanted sequences, for example by extraction of the RNA from cells producing high levels of the wanted gene and conversion of the RNA into the corresponding cDNA using the enzyme reverse transcriptase.

Alternatively, the suitable oligonucleotides specific for LGR6-SVs can be synthesised and used as primers for the amplification of LGR6-SVs cDNA fragments by RACE-PCR (M. A. Innis et al., PCR Protocols, A Guide to Methods and Applications, Academic Press, 1990).

The term “substantially purified” refers to molecules, either nucleic or amino acid sequences, that are removed from their natural environment, isolated or separated, and are at least 60% free, preferably 75% free, and most preferably, 90% free from other components with which they are naturally associated.

The term “hybridization” as used herein shall include any process by which a strand of nucleic acid joins with complementary strand through a base pairing (Coombs J, 1994, Dictionary of Biotechnology, Stokton Press, New York N.Y.). “Amplification” is defined as the production of additional copies of a nucleic acid sequence and is generally carried out using polymerase chain reaction technologies well known in the art (Dieffenbach and Dveksler, 1995, PCR Primer, a Laboratory Manual, Cold Spring Harbor Press, Plainview N.Y.).

“Stringency” typically occurs in a range from about Tm-5° C. (5° C. below the melting temperature of the probe) to about 20° C. to 25° C. below Tm.

The term “stringent conditions” refers to hybridization and subsequent washing conditions, which those of ordinary skill in the art conventionally refer to as “stringent”. See Ausubel et al., Current Protocols in Molecular Biology, Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

As used herein, stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:
Tm=81.5C+16.6(LogM)+0.41(% GC)−0.61(% form)−500/L
where M is the molarity of monovalent cations, % GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to the equation of Meinkoth, hybridization will occur even if there is up to about 10% mismatch.

As used herein, “highly stringent conditions” are those which provide a Tm which is not more than 10° C. below the Tm that would exist for a perfect duplex with the target sequence, either as calculated by the above formula or as actually measured. “Moderately stringent conditions” are those, which provide a Tm, which is not more than 20° C. below the Tm that would exist for a perfect duplex with the target sequence, either as calculated by the above formula or as actually measured. Without limitation, examples of highly stringent (5-10° C. below the calculated or measured Tm of the hybrid) and moderately stringent (15-20° C. below the calculated or measured Tm of the hybrid) conditions use a wash solution of 2×SSC (standard saline citrate) and 0.5% SDS (sodium dodecyl sulfate) at the appropriate temperature below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE (standard saline-phosphate-EDTA)), 5× Denhardt's reagent, 0.5% SDS, 100 &micro;g/ml denatured, fragmented salmon sperm DNA at a temperature approximately 20 to 25° C. below the Tm. If mixed probes are used, it is preferable to use tetramethyl ammonium chloride (TMAC) instead of SSC (Ausubel, 1987, 1999).

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present. The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “LGR6-SVs polypeptide” refers to a polypeptide comprising the amino acid sequence of any of SEQ ID NO: 2 and SEQ ID NO: 4.

The term “naturally occurring or “native” when used in connection with biological materials such as nucleic acid molecules, polypeptides, host cells, and the like, refers to materials which are found in nature and are not manipulated by man. Similarly, “non-naturally occurring or “non-native” as used herein refers to a material that is not found in nature or that has been structurally modified or synthesized by man.

The terms “effective amount’ and “therapeutically effective amount each refer to the amount of an LGR6-SVs polypeptide or LGR6-SVs nucleic acid molecule used to support an observable level of one or more biological activities of the LGR6-SVs polypeptides as set forth herein.

The term “pharmaceutically acceptable carrier” or “physiologically acceptable carrier” as used herein refers to one or more formulation materials suitable for accomplishing or enhancing the delivery of the LGR6-SVs polypeptide, LGR6-SVs nucleic acid molecule, or LGR6-SVs selective binding agent as a pharmaceutical composition.

The term “antigen” refers to a molecule or a portion of a molecule capable of being bound by a selective binding agent, such as an antibody, and additionally capable of being used in an animal to produce antibodies capable of binding to an epitome of that antigen. An antigen may have one or more epitomes.

The term “selective binding agent” refers to a molecule or molecules having specificity for an LGR6-SVs polypeptide. As used herein, the terms, “specific” and “specificity” refer to the ability of the selective binding agents to bind to human LGR6 SVS polypeptides and not to bind to human non-LGR6-SVs polypeptides. It will be appreciated, however, that the selective binding agents may also bind orthologs of the polypeptide as set forth in any of SEQ ID NO: 2 and SEQ ID NO: 4.

Nucleic Acid Molecules

The nucleic acid molecules encoding a polypeptide comprising the amino acid sequence of an LGR6-SVs polypeptide can readily be obtained in a variety of ways including, without limitation, chemical synthesis, cDNA or genomic library screening, expression library screening, and/or PCR amplification of cDNA.

Recombinant DNA methods used herein are generally those set forth In Sambrook et al., Molecular Cloning: A Laboratory Manual (Cold Spring Harbor Laboratory Press, 1989) and/or Current Protocols in Molecular Biology (Ausubel et al., eds., Green Publishers Inc. and Wiley and Sons 1994). The invention provides for nucleic acid molecules as described herein and methods for obtaining such molecules.

Where a gene encoding the amino acid sequence of an LGR6-SVs polypeptide has been identified from one species, all or a portion of that gene may be used as a probe to identify orthologs or related genes from the same species. The probes or primers may be used to screen cDNA libraries from various tissue sources believed to express the LGR6 SVS polypeptide. In addition, part or all of a nucleic acid molecule having the sequence as set forth in any of SEQ ID NO: 1 and SEQ ID NO: 3 may be used to screen a genomic library to identify and isolate a gene encoding the amino acid sequence of an LGR6-SVs polypeptide. Typically, conditions of moderate or high stringency will be employed for screening to minimize the number of false positives obtained from the screening.

Nucleic acid molecules encoding the amino acid sequence of LGR6-SVs polypeptides may also be identified by expression cloning which employs the detection of positive clones based upon a property of the expressed protein. Typically, nucleic acid libraries are screened by the binding an antibody or other binding partner (e.g., receptor or ligand) to cloned proteins that are expressed and displayed on a host cell surface. The antibody or binding partner is modified with a detectable label to identify those cells expressing the desired clone.

Recombinant expression techniques conducted in accordance with the descriptions set forth below may be followed to produce these polynucleotides and to express the encoded polypeptides. For example, by inserting a nucleic acid sequence that encodes the amino acid sequence of an LGR6-SVs polypeptide into an appropriate vector, one skilled in the art can readily produce large quantities of the desired nucleotide sequence. The sequences can then be used to generate detection probes or amplification primers.

Alternatively, a polynucleotide encoding the amino acid sequence of an LGR6-SVs polypeptide can be inserted into an expression vector. By introducing the expression vector into an appropriate host, the encoded LGR6-SVs polypeptide may be produced in large amounts.

Another method for obtaining a suitable nucleic acid sequence is the polymerase chain reaction (PCR). In this method, cDNA is prepared from poly (A)+RNA or total RNA using the enzyme reverse transcriptase. Two primers, typically complementary to two separate regions of cDNA encoding the amino acid sequence of an LGR6-SVs polypeptide, are then added to the cDNA along with a polymerase such as Taq polymerase, and the polymerase amplifies the cDNA region between the two primers.

Another means of preparing a nucleic acid molecule encoding the amino acid sequence of an LGR6-SVs polypeptide is chemical synthesis using methods well known to the skilled artisan such as those described by Engels et al., 1989, Angew. C11em. Ind. Ed. 28-716-34. These methods include, inter alia, the phosphotriester, phosphoramidite, and H-phosphonate methods for nucleic acid synthesis. A preferred is method for such chemical synthesis is polymer-supported synthesis using standard phosphoramidite chemistry. Typically, the DNA encoding the amino acid sequence of an LGR6-Vs polypeptide will be several hundred nucleotides in length. Nucleic acids larger than about 100 nucleotides can be synthesized as several fragments using these methods. The fragments can then be ligated together to form the full-length nucleotide sequence of an LGR6 SVS gene. Usually, the DNA fragment encoding the amino-terminus of the polypeptide will have an ATG, which encodes a methionine residue. This methionine may or may not be present on the mature form of the LGR6 SVS polypeptide, depending on whether the polypeptide produced in the host cell is designed to be secreted from that cell. Other methods known to the skilled artisan may be used as well.

In certain embodiments, nucleic acid variants contain codons which have been altered for optimal expression of an LGR6-SVs polypeptide in a given host cell. Particular codon alterations will depend upon the LGR6-SVs polypeptide and host cell selected for expression. Such “codon optimization’ can be carried out by a variety of methods, 3 c) for example, by selecting codons which are preferred for use in highly expressed genes in a given host cell. Computer algorithms, which incorporate codon frequency tables such as “Eco—high.Cod” for codon preference of highly expressed bacterial genes, may be used and are provided by the University of Wisconsin Package Version 9.0 (Genetics Computer Group, Madison, W1). Other useful codon frequency tables include “C. elegans—high.cod,” “C. elegans—low.cod,” “Drosophila—high.cod,” Human—high.cod . . . Maize—high.cod,” and “Yeast—high.cod.”

In some cases, it may be desirable to prepare nucleic acid molecules encoding LGR6-SVs polypeptide variants. Nucleic acid molecules encoding variants may be produced using site directed mutagenesis, PCR amplification, or other appropriate methods, where the primer(s) have the desired point mutations (see Sambrook et al., supra, and Ausubel et al., supra, for descriptions of mutagenesis techniques). Chemical synthesis using methods described by Engels et al., supra, may also be used to prepare such variants. Other methods known to the skilled artisan may be used as well.

Vectors and Host Cells

A nucleic acid molecule encoding the amino acid sequence of an LGR6-SVs polypeptide is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of an LGR6-SVs polypeptide may be amplified/expressed in prokaryotic, yeast, insect (baculovirus systems) and/or eukaryotic host cells. Selection of the host cell will depend in part on whether an LGR6-SVs polypeptide is to be post-translationally modified (e.g., glycosylated and/or phosphorylated). If so, yeast, insect, or mammalian host cells are preferable. For a review of expression vectors, see Meth. Enz., vol. 185 (D. V. Goeddel, ed., Academic Press 1990).

Typically, expression vectors used in any of the host cells will contain sequence for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the LGR6-SVs polypeptide coding sequence; the oligonucleotide sequence encodes polyHis (such as hexaHis), or another “tag” such as FLAG, HA (hemaglutinin influenza virus), or mye for which commercially available antibodies exist. This tag is typically fused to the polypeptide upon expression of the polypeptide, and can serve as a means for affinity purification of the LGR6-Vs polypeptide from the host cell. Affinity purification can be accomplished, for example, by column chromatography using antibodies against the tag as an affinity matrix. Optionally, the tag can subsequently be removed from the purified LGR6-SVs polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), or synthetic, or the flanking sequences may be native sequences that normally function to regulate LGR6-SVs polypeptide expression. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein—other than the LGR6-SVs gene flanking sequences—will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with a suitable oligonucleotide and/or flanking sequence fragment from the same or another species.

Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of an LGR6-SVs polypeptide. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various origins (e.g., SV40, polyoma, is adenovirus, vesicular stomatitis virus (VSV), or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide-coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene that will be expressed.

Amplification is the process wherein genes that are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure wherein only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes an LGR6-SVs polypeptide. As a result, increased quantities of LGR6-SVs polypeptide are synthesized from the amplified DNA.

A ribosome-binding site is usually necessary for translation initiation of mRNA and is characterized by a Shine-Dalgamo sequence (prokaryotes) or a Kozak sequence (eukaryotes). The element is typically located 3′ to the promoter and & to the coding sequence of an LGR6 SVS polypeptide to be expressed. The Shine-Dalgamo sequence is varied but is typically a polypurine (i.e., having a high A-G content). Many Shine-Dalgamo sequences have been identified, each of which can be readily synthesized using methods set forth herein and used in a prokaryotic vector.

A leader, or signal, sequence may be used to direct an LGR6-SVs polypeptide out of the host cell. Typically, a nucleotide sequence encoding the signal sequence is positioned in the coding region of an LGR6-SVs nucleic acid molecule, or directly at the 5′ end of an LGR6-SVs polypeptide-coding region. Many signal sequences have been identified, and any of those that are functional in the selected host cell may be used in conjunction with an LGR6-SVs nucleic acid molecule. Therefore, a signal sequence may be homologous (naturally occurring) or heterologous to the LGR6-SVs nucleic acid molecule. Additionally, a signal sequence may be chemically synthesized using methods described herein. In most cases, the secretion of an LGR6-SVs polypeptide from the host cell via the presence of a signal peptide will result in the removal of the signal peptide from the secreted LGR6-SVs polypeptide. The signal sequence may be a component of the vector, or it may be a part of an LGR6-SVs nucleic acid molecule that is inserted into the vector.

Included within the scope of this invention is the use of either a nucleotide sequence encoding a native LGR6-SVs polypeptide signal sequence joined to an LGR6-SVs polypeptide coding region or a nucleotide sequence encoding a heterologous signal sequence joined to an LGR6-SVs polypeptide coding region. The heterologous signal sequence selected should be one that is recognized and processed, i.e., cleaved by a signal peptidase, by the host cell. For prokaryotic host cells that do not recognize and process the native LGR6-SVs polypeptide signal sequence, the signal sequence is substituted by a prokaryotic signal sequence selected, for example, from the group of the alkaline phosphatase, penicillinase, or heat-stable enterotoxin 11 leaders. For yeast secretion, the native LGR6-SVs polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a is particular signal peptide, or add pro-sequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired LGR6-SVs polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the LGR6 SVS gene especially where the gene used is a full-length genomic sequence or a fragment thereof. Where the intron is not naturally occurring within the gene (as for most cDNAs), the intron may be obtained from another source. The position of the intron with respect to flanking sequences and the LGR6 SVS gene is generally important, as the intron must be transcribed to be effective. Thus, when an LGR6-SVs cDNA molecule is being transcribed, the preferred position for the intron is 3′ to the transcription start site and 5′ to the poly-A transcription termination sequence. Preferably, the intron or introns will be located on one side or the other (i.e., 5′, or Y) of the cDNA such that it does not interrupt the coding sequence. Any intron from any source, including Viral, prokaryotic and eukaryotic (plant or animal) organisms, may be used to practice this invention, provided that it is compatible with the host cell into which it is inserted. Also included herein are synthetic introns. Optionally, more than one intron may be 5 used in the vector.

The expression and cloning vectors of the present invention will typically contain a promoter that is recognized by the host organism and operably linked to the molecule encoding the LGR6-Vs polypeptide. Promoters are untranscribed sequences located upstream (i.e., Y) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding LGR6-SVs polypeptide by removing the promoter from the source DNA by restriction enzyme digestion and inserting the desired promoter sequence into the vector. The native LGR6-SVs promoter sequence may be used to direct amplification and/or expression of an LGR6-SVs nucleic acid molecule. A heterologous promoter is preferred, however, if it pen-nits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase; a tryptophan (trp) promoter system; and hybrid promoters such as the tac promoter. Other known bacterial promoters are also suitable. Their sequences have been published, thereby enabling one skilled in the art to ligate them to the desired DNA sequence, using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest in controlling LGR6 SVS gene expression include, but are not limited to: the SV40 early promoter region (Bemoist and Chambon, 198 1, Nature 290:304-10); the CW promoter; the promoter contained in the 3′ long terminal repeat of Rous sarcoma virus (Yamamoto, et al., 1980, Cell 22:787-97); the herpes thymidine kinase promoter (Wagner et al., 1981, Proc. Natl. Acad. Sci. U.S.A. 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. U.S.A, 75:3727-31); or the tae promoter (DeBoer et al., 1983, Proc. Natl. Acad. Sci. U.S.A., 80:21-25). Also of interest are the following animal transcriptional control regions, is which exhibit tissue specificity and have been utilized in transgenic animals: the elastase I gene control region which is active in pancreatic cells (Swift et al., 1984, Cell 38:639-46; Omitz et al., 1986, Cold Spring Barbor Symp. Quant Biol. 50:399-409 (1986); MacDonald, 1987, Hepatology 7:425-515); the insulin gene control region which is active in pancreatic beta cells (Hanahan, 1985, Nature 315:115-22); the immunoglobulin gene control region which is active in lymphoid cells (Grosschedl et al., 1984, Cell 38:647-58; Adames et al., 1985, Nature 318:533 38; Alexander et al., 1987, Mol. Cell. Biol., 7:1436-44); the mouse mammary tumor virus control region which is active in testicular, breast, lymphoid and mast cells (Leder et al., 1986, Cell 45:485-95); the albumin gene control region which is active in liver (Pinkert et al., 1987, Genes and Devel. 1:268-76); the alpha-feto-protein gene control region which is active in liver (Krumlauf et al., 1985, Mol. Cell. Biol., 5:1639 48; Hammer et al., 1987, Science 235:53-58); the alpha 1-antitrypsin gene control region which is active in the liver (Kelsey et al., 1987, Genes and Devel. 1: 161-71); the beta-globin gene control region which is active in myeloid cells (Mogram et al., 1985, Nature 315:338-40; Kollias et al., 1986, Cell 46:89-94); the myelin basic protein gene control region which is active in oligodendrocyte cells in the brain (Readhead et al., 1987, Cell 48:703-12); the myosin light chain-2 gene control region which is active in skeletal muscle (Sani, 1985, Nature 314:283-86); and the gonadotropic releasing hormone gene control region which is active in the hypothalamus (Mason et al., 1986, Science 234:1372-78).

An enhancer sequence may be inserted into the vector to increase the transcription of a DNA encoding an LGR6-SVs polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to an LGR6-SVs nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

Preferred vectors for practicing this invention are those that are compatible with bacterial, insect, and mammalian host cells. Such vectors include, inter alia, pCRII, pCR3, and pcDNA3.1 (Invitrogen, San Diego, Calif.), pBSII (Stratagene, La Jolla, Calif.), pET 15 (Novagen, Madison, W1), pGEX (Pharmacia Biotech, Piscataway, N.J.), pEGFP-N2 (Clontech, Palo Alto, Calif.), pETL (BlueBaell, Invitrogen), pDSRalpha (PCT Pub. No. WO 90/14363) and pFastBacDual (Gibco-BRL, Grand Island, N.Y.).

Additional suitable vectors include, but are not limited to, cosmids, plasmids, or modified viruses, but it will be appreciated that the vector system must be compatible with the selected host cell. Such vectors include, but are not limited to plasmids such as Bluescript plasmid derivatives (a high copy number ColEl-based phagemid; Stratagene Cloning Systems, La Jolla Calif.), PCR cloning plasmids designed for cloning Taq-amplified PCR products (e.g., TOPOTm TA Cloning′ Kit and PCR2.1″ plasmid derivatives; invitrogen), and mammalian, yeast or virus vectors such as a baculovirus expression system (pBacPAK plasmid derivatives; Clontech).

After the vector has been constructed and a nucleic acid molecule encoding an LGR6-SVs polypeptide has been inserted into the proper site of the vector, the completed vector may be inserted into a suitable host cell for amplification and/or polypeptide expression. The transformation of an expression vector for an LGR6-Vs polypeptide into a selected host cell may be accomplished by well-known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast, insect, or vertebrate cell). The host cell, when cultured under appropriate conditions, synthesizes an LGR6-SVs polypeptide that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

A number of suitable host cells are known in the art and many are available from the American Type Culture Collection (ATCC), Manassas, Va. Examples include, but are not limited to, mammalian cells, such as Chinese hamster ovary cells (CHO), CHO MFR(−) cells (Urlaub et al., 1980, Proc. Natl. Acad. Sci. U.SA. 97:4216-20), human embryonic kidney (HEK) 293 or 293T cells, or 3T3 cells. The selection of suitable mammalian host cells and methods for transformation, culture, amplification, screening, product production, and purification are known in the art. Other suitable mammalian cell lines are the monkey COS-1 and COS-7 cell lines, and the CV-1 cell line. Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-e or N1H mice, BHY, or HaK hamster cell lines. Each of these cell lines is known by and available to those skilled in the art of protein expression.

Similarly useful as host cells suitable for the present invention are bacterial cells. For example, the various strains of E. coli (e.g., HB101, DH5α, DH10, and MC1061) are well known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastoris.

Additionally, where desired, insect cell systems may be utilized in the is methods of the present invention. Such systems are described, for example, in Kitts et al., 1993, Biotechniques, 14:810-17; Lucklow, 1993, Curr. Opin. Biotechnol. 4:564-72; and Lucklow et al., 1993, J. Virol., 67:4566-79. Preferred insect cells are Sf-9 and H3 (Invitrogen).

One may also use transgenic animals to express glycosylated LGR6-SVs polypeptides. For example, one may use a transgenic milk-producing animal (a cow or goat, for example) and obtain the present glycosylated polypeptide in the animal milks. One may also use plants to produce LGR6-SVs polypeptides, however. In general, the glycosylation occurring in plants is different from that produced in mammalian cells, and may result in a glycosylated product which is not suitable for human therapeutic use. Polypeptide Production Host cells comprising an LGR6-SVs polypeptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with scram and/or growth factors as necessary for the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

Purification and Isolation of the Polypeptide

The amount of an LGR6-SVs polypeptide produced by a host cell can be evaluated using standard methods known in the art. Such methods include, without limitation Western blot analysis, SDS-polyacrylamide gel electrophoresis, non-denaturing gel is electrophoresis, High Performance Liquid Chromatography (HPLC) separation, immunoprecipitation, and/or activity assays such as DNA binding gel shift assays.

If an LGR6-SVs polypeptide has been designed to be secreted from the host cells, the majority of polypeptide may be found in the cell culture medium. If however, the LGR6-SVs polypeptide is not secreted from the host cells. It will be present in the cytoplasm and/or the nucleus (for eukaryotic host cells) or in the cytosol (for gram negative bacteria host cells).

For an LGR6-SVs polypeptide situated in the host cell cytoplasm and/or nucleus (for eukaryotic host cells) or in the cytosol (for bacterial host cells), the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If an LGR6-SVs polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The solubilized LGR6-SVs polypeptide can then be analyzed using gel electrophoresis, immunoprecipitation, or the like. If it is desired to isolate the LGR6-SVs polypeptide, isolation may be accomplished using standard methods such as those described herein and in Marston et al., 1990, Meth. Enz., 182:264-75.

In some cases, an LGR6-SVs polypeptide may not be biologically active upon isolation. Various methods for “refolding” or converting the polypeptide to its tertiary structure and generating disulfide linkages can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential is allowing for disulfide shuffling to occur in the formation of the protein's cysteine bridges. Some of the commonly used redox couples include cysteine/cystamine, glutathione (GSH)/dithiols GSH, cupric chloride, dithiothreitol (DTT)/dithiane DTT, and 2-2-mercaptoethanol (bME)/dithio-b (ME). In many instances, a cosolvent may be used or may be needed to increase the efficiency of the refolding, and the more common reagents used for this purpose include glycerol, polyethylene glycol of various molecular weights, arginine and the like.

If inclusion bodies are not formed to a significant degree upon expression of an LGR6-SVs polypeptide, then the polypeptide will be found primarily in the supernatant after centrifugation of the cell homogenate. The polypeptide may be further isolated from the supernatant using methods such as those described herein.

The purification of an LGR6-SVs polypeptide from solution can be accomplished using a variety of techniques. If the polypeptide has been synthesized such that it contains a tag such as Hexa-histidine (LGR6-SVs polypeptide/hexaHis) or other small peptide such as FLAG (Eastman Kodak Co., New Haven, Conn.) or myc (invitrogen) at either its carboxyl- or amino-terminus, it may be purified in a one-step process by passing the solution through an affinity column where the column matrix has a high affinity for the tag. For example, poly-histidine binds with great affinity and specificity to nickel. Thus, an affinity column of nickel (such as the Qiagen nickel columns) can be used for purification of LGR6-SVs polypeptide/polyHis. See, e.g., Current Protocols in Molecular Biology 10.11.8 (Ausubel et al., eds., Green Publishers Inc. and Wiley and Sons 1993).

Additionally, LGR6-SVs polypeptides may be purified through the use of a monoclonal antibody that is capable of specifically recognizing and binding to an LGR6-SVs polypeptide.

Other suitable procedures for purification include, without limitation, affinity chromatography, immunoaffinity chromatography, ion exchange chromatography, molecular sieve chromatography, HPLC, electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

LGR6-SVs polypeptides may also be prepared by chemical synthesis methods is (such as solid phase peptide synthesis) using techniques known in the art such as those set forth by Merrifield et al., 1963, J Am. Chem. Soc. 85:2149; Houghten et al., 1985, Proc Natl Acad. Sci. USA 82:5132; and Stewart and Young, Solid Phase Peptide Synthesis (Pierce Chemical Co. 1984). Such polypeptides may be synthesized with or without a methionine on the amino-terminus. Chemically synthesized LGR6-SVs polypeptides may be oxidized using methods set forth in these references to form disulfide bridges. Chemically synthesized LGR6-SVs polypeptides are expected to have comparable biological activity to the corresponding LGR6-SVs polypeptides produced recombinantly or purified from natural sources, and thus may be used interchangeably with a recombinant or natural LGR6-SVs polypeptide.

Another means of obtaining LGR6-SVs polypeptide is via purification from biological samples such as source tissues and/or fluids in which the LGR6-SVs polypeptide is naturally found. Such purification can be conducted using methods for protein purification as described herein. The presence of the LGR6-SVs polypeptide during purification may be monitored, for example, using an antibody prepared against recombinantly produced LGR6-SVs polypeptide or peptide fragments thereof. A number of additional methods for producing nucleic acids and polypeptides are known in the art, and the methods can be used to produce polypeptides having specificity for LGR6-SVs polypeptide. See, e.g., Roberts et al., 1997, Proc. Natl. Acad. Sci. U.S.A. 94:12297-303, which describes the production of fusion proteins between an mRNA and its encoded peptide. See also, Roberts, 1999, Curr. Opin. Chem. Biol. 3:268-73. Additionally, U.S. Pat. No. 5,824,469 describes methods for obtaining oligonucleotides capable of carrying out a specific biological function. The procedure involves generating a heterogeneous pool of oligonucleotides, each having a 5, randomized sequence, a central preselected sequence, and a 3′ randomized sequence. The resulting heterogeneous pool is introduced into a population of cells that do not exhibit the desired biological function. Subpopulations of the cells are then screened for those that exhibit a predetermined biological function. From that subpopulation, oligonucleotides capable of carrying out the desired biological function are isolated.

U.S. Pat. Nos. 5,763,192; 5,814,476; 5,723,323; and 5,817,483 describe processes for producing peptides or polypeptides. This is done by producing stochastic genes or fragments thereof, and then introducing these genes into host cells, which produce one or more proteins, encoded by the stochastic genes. The host cells are then screened to identify those clones producing peptides or polypeptides having 1.5 the desired activity.

Another method for producing peptides or polypeptides is described in PCT/IJS98/20094 (WO99/15650) filed by Athersys, Inc. Known as “Random Activation of Gene Expression for Gene Discovery” (RAGE-GD), the process involves the activation of endogenous gene expression or over-expression of a gene by in situ recombination methods. For example, expression of an endogenous gene is activated or increased by integrating a regulatory sequence into the target cell that is capable of activating expression of the gene by non-homologous or illegitimate recombination. The target DNA is first subjected to radiation, and a genetic promoter inserted. The promoter eventually locates a break at the front of a gene, initiating transcription of the gene. This results in expression of the desired peptide or polypeptide.

It will be appreciated that these methods can also be used to create comprehensive LGR6-SVs polypeptide expression libraries, which can subsequently be used for high throughput phenotypic screening in a variety of assays, such as biochemical assays, cellular assays, and whole organism assays (e.g., plant, mouse, etc.) synthesis. It will be appreciated by those skilled in the art that the nucleic acid and polypeptide molecules described herein may be produced by recombinant and other means.

Selective Binding Agents

The term “selective binding agent” refers to a molecule that has specificity for one or more LGR6-SVs polypeptides. Suitable selective binding agents include, but are not limited to, antibodies and derivatives thereof, polypeptides, and small molecules. Suitable selective binding agents may be prepared using methods known in the art.

An exemplary LGR6-SVs polypeptide selective binding agent of the present invention is capable of binding a certain portion of the LGR6-SVs polypeptide thereby inhibiting the binding of the polypeptide to an LGR6-SVs polypeptide receptor.

Selective binding agents such as antibodies and antibody fragments that bind LGR6-SVs polypeptides are within the scope of the present invention. The antibodies may is be polyclonal including monospecific polyclonal; monoclonal (MAbs); recombinant; chimeric; humanized, such as complementarity-determining region (CDR)-grafted; human; single chain; and/or bispecific; as well as fragments; variants; or derivatives thereof. Antibody fragments include those portions of the antibody that bind to an epitope on the LGR6-SVs polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward an LGR6-SVs polypeptide generally are produced in animals (e.g., rabbits or mice) by means of multiple subcutaneous or intraperitoneal injections of LGR6-SVs polypeptide and an adjuvant it may be useful to conjugate an LGR6-SVs polypeptide to a carrier protein that is immunogenic in the species to be immunized, such as keyhole limpet hemocyanin, scrum, albumin, bovine thyroglobulin, or soybean trypsin inhibitor. Also, aggregating agents such as alum are used to enhance the immune response. After immunization, the animals are bled and the serum is assayed for anti-LGR6-SVs antibody titer.

Monoclonal antibodies directed toward LGR6-SVs polypeptides are produced using any method that provides for the production of antibody molecules by continuous cell lines in culture. Examples of suitable methods for preparing monoclonal antibodies include the hybridoma methods of Kohler et al., 1975, Nature 256:495-97 and the human B-cell hybridoma method (Kaozbor, 1984, J. Immunol. 133:3001; Brodeur et al., Monoclonal Antibody Production Techniques and Applications 51-63 (Marcel Dekker, Inc., 1987). Also provided by the invention are hybridoma cell lines that produce monoclonal antibodies reactive with LGR6-SVs polypeptides.

Monoclonal antibodies of the invention may be modified for use as therapeutics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody of the invention is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody

Cell Source Identification Using LGR6-SVs Polypeptide

In accordance with certain embodiments of the invention, it may be useful to be able to determine the source of a certain cell type associated with an LGR6-SVs polypeptide. For example, it may be useful to determine the origin of a disease or pathological condition as an aid in selecting an appropriate therapy. In certain embodiments, nucleic acids encoding an LGR6-SVs polypeptide can be used as a probe to identify cells described herein by screening the nucleic acids of the cells with such a probe. In other embodiments, one may use aliti-LGR6-SVs polypeptide antibodies to test for the presence of LGR6-SVs polypeptide in cells, and thus, determine if such cells are of the types described herein.

LGR6-SVs Polypeptide Compositions and Administration

Therapeutic compositions are within the scope of the present invention. Such LGR6-SVs polypeptide pharmaceutical compositions may comprise a therapeutically effective amount of an LGR6-SVs polypeptide or an LGR6-SVs nucleic acid molecule in admixture with a pharmaceutically or physiologically acceptable formulation agent selected for suitability with the mode of administration. Pharmaceutical compositions may comprise a therapeutically effective amount of one or more LGR6-SVs polypeptide selective binding agents in admixture with a pharmaceutically or physiologically acceptable formulation agent selected for suitability with the mode of administration.

Acceptable formulation materials preferably are nontoxic to recipients at the dosages and concentrations employed.

The pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropylbeta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counter ions (such as sodium), preservatives (such as benzalkonium chloride, benzoic acid, salicylic acid, phenethyl alcohol, methylparaben, propylparaben, chlorhexidine, sorbic acid, or hydrogen peroxide), 5 solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See Reinington's Pharmaceutical Sciences (18th Ed., AR. Gennaro, ed., Mack Publishing Company 1990).

The optimal pharmaceutical composition will be determined by a skilled artisan depending upon, for example, the intended route of administration, delivery format, and desired dosage. See, e.g., Remington's Pharmaceutical Sciences, supra. Such compositions may influence the physical state, stability, rate of in vivo release, and rate of in vivo clearance of the LGR6-SVs molecule.

The primary vehicle or carrier in a pharmaceutical composition may be either aqueous or non-aqueous in nature. For example, a suitable vehicle or carrier for injection may be water, physiological saline solution, or artificial cerebrospinal fluid, possibly supplemented with other materials common in compositions for parenteral administration. Neutral buffered saline or saline mixed with serum albumin are further exemplary vehicles.

Other exemplary pharmaceutical compositions comprise Tris buffer of about pH 7.0-8.5, or acetate buffer of about pH 4.05.5, which may further include sorbitol or a suitable substitute. In one embodiment of the present invention, LGR6-Vs polypeptide compositions may be prepared for storage by mixing the selected composition having the desired degree of purity with optional formulation agents (Remington's Pharmaceutical Sciences, supra) in the form of a lyophilized cake or an aqueous solution. Further, the LGR6-SVs polypeptide product may be formulated as a lyophilizate using appropriate excipients such as sucrose.

The LGR6-SVs polypeptide pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention may be in the form of a pyrogen-free, parenterally acceptable, aqueous solution comprising the desired LGR6-SVs molecule in a pharmaceutically acceptable vehicle. A particularly suitable vehicle for parenteral injection is sterile distilled water in which an LGR6-SVs molecule is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, LGR6-SVs polypeptide may be formulated as a dry powder for inhalation. LGR6-SVs polypeptide or nucleic acid molecule inhalation solutions may also be formulated with a propellant for aerosol delivery. In yet another embodiment, solutions may be nebulized. Pulmonary administration is further described in PCT Pub. No. WO 94/20069, which describes the pulmonary delivery of chemically modified proteins.

It is also contemplated that certain formulations may be administered orally. In one embodiment of the present invention, LGR6-SVs polypeptides that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the LGR6-SVs polypeptide. Diluents, flavorings, low melting point waxes, vegetable oils, lubricants, suspending agents, tablet disintegrating agents, and binders may also be employed.

Another pharmaceutical composition may involve an effective quantity of LGR6-SVs polypeptides in a mixture with lion-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or tale.

Additional LGR6-SVs polypeptide pharmaceutical compositions will be evident to those skilled ill the art, including formulations involving LGR6-SVs polypeptides in sustained- or controlled-delivery formulations. Techniques for formulating a variety of other sustained- or controlled-delivery means, such as liposome carriers, bio erodible microparticles or porous beads and depot injections, are also known to those skilled in the art. See, e.g., PCT/US93/00829, which describes the controlled release of porous polymeric microparticles for the delivery of pharmaceutical compositions.

Additional examples of sustained-release preparations include semipermeable polymer matrices in the form of shaped articles, e.g. films, or microcapsules.

Sustained release matrices may include polyesters, hydrogels, polylactides (U.S. Pat. No. 3,773,919 and European Patent No. 058481), copolymers of L-glutamic acid and gamma ethyl-L-glutamate (Sidman et al., 1983, Biopolymers 22:547-56), poly(2-hydroxyethyl-methacrylate) (Langer et al., 1981, J Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent No. 133988). Sustained-release compositions may also include liposomes, which can be prepared by any of several methods known in the art. See, e.g., Eppstein et al., 1985, Proc. Natl. Acad. Sci. USA 82:3688-92; and European Patent Nos. 036676, 088046, and 143949.

The LGR6-SVs pharmaceutical composition to be used for in vivo administration typically must be sterile. This may be accomplished by filtration through sterile filtration membranes. Where the composition is lyophilized, sterilization using this method may be conducted either prior to, or following, lyophilization and reconstitution. The composition for parenteral administration may be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration. In a specific embodiment, the present invention is directed to kits for producing a single-dose administration unit. The kits may each contain both a first container having a dried protein and a second container having an aqueous formulation. Also included within the scope of this invention are kits containing single and mufti-chambered pre-filled syringes (e.g., liquid syringes and lyosyringes).

The effective amount of an LGR6-SVs pharmaceutical composition to be employed therapeutically will depend, for example, upon the therapeutic context and objectives.

One skilled in the art will appreciate that the appropriate dosage levels for treatment will thus vary depending. In part, upon the molecule delivered, the indication for which the LGR6-Vs molecule is being used, the route of administration, and the size (body weight, body surface, or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. In other embodiments, the dosage may range from 0.1 mg/kg up to about 100 mg/kg; or 1 g/kg up to about 100 mg/kg; or 5 g/kg up to about 100 mg/kg.

The frequency of dosing will depend upon the pharmacokinetic parameters of the LGR6-SVs molecule in the formulation being used. Typically, a clinician will administer the composition until a dosage is reached that achieves the desired effect.

The composition may therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions may be administered by bolus injection or continuously by infusion, or by implantation device.

Alternatively or additionally, the composition may be administered locally via implantation of a membrane, sponge, or other appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use LGR6-SVs polypeptide pharmaceutical compositions in an ex vivo mariner. In such instances, cells, tissues, or organs that have been removed from the patient are exposed to LGR6-SVs polypeptide pharmaceutical compositions after which the cells, tissues, or organs are subsequently implanted back into the patient.

In other cases, an LGR6-SVs polypeptide can be delivered by implanting certain cells that have been genetically engineered, using methods such as those described herein, to express and secrete the LGR6-SVs polypeptide. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patients immune system or by other detrimental factors from the surrounding tissues.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally-silent LGR6-Vs gene, or an under-expressed gene, and thereby produce a cell, which expresses therapeutically efficacious amounts of LGR6-SVs polypeptides.

Homologous recombination is a technique originally developed for targeting genes to induce or correct mutations in transcriptionally active genes. Kucheriapati, 1989, Prog. in Nucl. Add Res. & Mol. Biol. 36:301. The basic technique was developed as a method for introducing specific mutations into specific regions of the mammalian genome (Thomas et al., 1986, Cell 44:419-28; Thomas and Capecchi, 1987, Cell 51:503-12; Doetschman et al., 1988, Proc. Natl. Acad. Sci. U.S.A. 85:858387) or to correct specific mutations within defective genes (Doetschman et al., 1987, Nature 330:576-78). Exemplary homologous recombination techniques are described in U.S. Pat. No. 5,272,071; European Patent Nos. 9193051 and 505500; PCT/US90/07642, and PCT Pub No. WO 91/09955).

Through homologous recombination, the DNA sequence to be inserted into the genome can be directed to a specific region of the gene of interest by attaching it to targeting DNA. The targeting DNA is a nucleotide

Therapeutic Uses

LGR6-SVs nucleic acid molecules. polypeptides, and agonists and antagonists thereof can be used to treat, diagnose, ameliorate, or prevent a number of diseases, disorders, or conditions, including those recited herein.

LGR6-SVs polypeptide agonists and antagonists include those molecules which regulate LGR6-SVs polypeptide activity and either increase or decrease at least one activity of the mature form of the LGR6-SVs polypeptide. Agonists or antagonists may be co-factors, such as a protein, peptide, carbohydrate, lipid, or small molecular weight molecule, which interact with LGR6-SVs polypeptide and thereby regulate its activity.

Potential polypeptide agonists or antagonists include antibodies that react with either soluble or membrane-bound forms of LGR6-SVs polypeptides that comprise part or all of the extracellular domains of the said proteins. Molecules that regulate LGR6-SVs polypeptide expression typically include nucleic acids encoding LGR6-SVs polypeptide 5 that can act as anti-sense regulators of expression.

Seven members of the glycoprotein hormone receptor subfamily have been previously identified. Among these are thyroid stimulating hormone (TSH) receptor, follicle stimulating hormone (FSH) receptor, luteinizing hormone (LH)/chorionic gonadotropin (CG) receptor, and leucine-rich repeat-containing G-protein coupled receptor (LGR) 4, LGR5, LGR6, LGR7 and LGR8. The TSH, FSH, and LH/CG receptors have been well characterized functionally, with signaling through these receptors playing an important role in the proliferation and differentiation of the thyroid gland and the gonads. Signaling through the TSH receptor is known to influence basal metabolism by regulating the production of thyroid hormones. Autoimmune is antibodies against epitopes of the N-terminal extracellular domain of the TSH receptor cause various thyroid hormone-associated metabolic disorders. Such autoimmune antibodies can be antagonistic and cause a form of hypothyroidism (a subgroup of Hashimoto's thyroiditis) or can be agonistic and cause a form of hyperthyroidism (Graves' disease). Signaling through the FSH and LH/CG receptors is known to play a critical role in the maintenance of reproductive function in males and females (i.e., gonadal maturation and gonadal steroid production).

Additionally, signaling through the LH/CG receptor is known to play an important role in the maintenance of pregnancy by stimulating the corpus luteum to produce steroid hormones during the first trimester. Because the TSH, FSH, and LH/CG receptors are known to have important developmental (i.e., proliferation and differentiation) and physiological functions, it is likely that LGR6-SVs also plays an important role in development and in human physiology.

As stated in example 3, LGR6 is part of a LGR subgroup composed of LGR4, LGR5 and LGR6. Yamamoto et al. suggest that GPR49 (LGR5) may be critically involved in the development of hepatocellular carcinoma (HCC). They also suggest that GPR49 is a β-catenin target, the expression of Gpr49 being up-regulated by β-catenin mutation (Yamamoto Y. et al. 2003. Hepatology. 37:528-533). β-catenin is involved in carcinogenesis through the activation of the Wnt-signaling pathway, can act as a coactivator of certain transcriptional factors, such as Tcf/LEF and CREB, subsequently resulting in the activation of downstream target genes such as c-myc, cyclin D1, MDR1, WISP1 and thus likely Gpr49. β1-catenin is involved in a large variety of cancers including adenomatous polyposis, malignant transformation of epithelial cells, colorectal cancer, breast cancer cells, retinoblastoma, colorectal tumorigenesis, melanoma, ovarian carcinomas, endometrioid tumors, Hepatoblastoma, sporadic medulloblastomas, lung cancers, blastoma, mesotheliomas, small intestinal adenocarcinoma, colorectal neoplasia, pilomatricoma, hair tumors (see OMIM on β1-catenin, http://www.ncbi.nim.nih.gov/entrez/dispomim.cgi?id=116806). In addition to the role of β1-catenin and LGR5 in cancer, mutations in the hinge-conserved regions of glycoprotein hormone receptors can result in the constitutive activation of the receptor leading to carcinogenesis or congenital disorders (constitutively activating mutant of TSHR causes hyperfunctioning thyroid adenomas, Yamamoto et al.; see also example 3). Based on the above comments, it is suggested that LGR6 is also critical in the development of cancers. As such, LGR6-SVs nucleic acid molecules, polypeptides, and agonists and antagonists thereof may be useful in diagnosing or treating cancers. Examples of such cancers include, but are not limited to, hepatocellular carcinoma, adenomatous polyposis, malignant transformation of epithelial cells, colorectal cancer, breast cancer cells, retinoblastoma, colorectal tumorigenesis, melanoma, ovarian carcinomas, endometrioid tumors, Hepatoblastoma, sporadic medulloblastomas, lung cancers, blastoma, mesotheliomas, small intestinal adenocarcinoma, colorectal neoplasia, pilomatricoma, hair tumors. In particular, antagonists of LGR6.1 (e.g. antibodies targeted to LGR6.1) may be useful in the diagnosis or treatment of cancer. Furthermore, mutations in the hinge-conserved sequences of LGR6.1 (or LGR6) can be corrected for the treatment of cancer, congenital disorders and of the conditions and diseases mentioned hereafter, but are not limited to. LGR6.2, being a secreted protein, might function as a LGR6.1 antagonist. As such LGR6.2 or agonists thereof may be useful in the diagnosis or treatment of cancer, or even congenital disorders, and of the conditions or diseases mentioned hereafter, but are not limited to.

Since LGR6-SVs polypeptide expression has been detected in skeletal muscle, LGR6-SVs nucleic acid molecules, polypeptides, and agonists and antagonists thereof may be useful in diagnosing or treating diseases and conditions affecting skeletal muscle. Examples of such diseases and conditions include, but are not limited to, cachexia and muscular dystrophy. Other diseases and conditions associated with skeletal muscle development and function are encompassed within the scope of this invention.

Since LGR6-SVs polypeptide expression has been detected in the uterus, LGR6-SVs nucleic acid molecules, polypeptides, and agonists and antagonists thereof may be useful in diagnosing or treating diseases and conditions affecting skeletal muscle. Examples of such diseases and conditions include, but are not limited to, miscarriage, endometriosis, uterine cancer, and female infertility. Other diseases and conditions associated with uterine development and function are encompassed within the scope of this invention.

Since LGR6-SVs polypeptide expression has been detected in the adrenal gland, LGR6-SVs nucleic acid molecules, polypeptides, and agonists and antagonists thereof may be useful in diagnosing or treating diseases and conditions affecting the adrenal gland. Examples of such diseases and conditions include, but are not limited to, Cushing's disease and Addison's disease. Other diseases and conditions associated with the development and function of the adrenal gland are encompassed within the scope of this invention.

Since LGR6-SVs polypeptide expression has been detected in the testes, LGR6-SVs nucleic acid molecules, polypeptides, and agonists and antagonists thereof may be useful in diagnosing or treating diseases and conditions affecting the testes. Examples of such diseases and conditions include, but are not limited to, male infertility and testicular carcinoma. Other diseases and conditions associated with the development and function of the testes are encompassed within the scope of this invention.

Since LGR6-SVs polypeptide expression has been detected in the bone marrow, LGR6-SVs nucleic acid molecules, polypeptides, and agonists and antagonists thereof may be useful in diagnosing or treating diseases and conditions affecting the bone marrow. Examples of such diseases and conditions include, but are not limited to, leukemia.

Other diseases and conditions associated with the development and function of the bone marrow are encompassed within the scope of this invention.

Since LGR6-SVs polypeptide expression has been detected in the fetal kidney, LGR6-SVs nucleic acid molecules, polypeptides, and agonists and antagonists thereof may be useful in diagnosing or treating diseases and conditions affecting the kidney.

Examples of such diseases and conditions include, but are not limited to, anemia, hypertension, and low blood pressure. Other diseases and conditions associated with the development and function of the kidney are encompassed within the scope of this invention.

Since LGR6-SVs polypeptide expression has been detected in the ovary, LGR6-SVs nucleic acid molecules, polypeptides, and agonists and antagonists thereof may be useful in diagnosing or treating diseases and conditions affecting the ovaries. Examples of such diseases and conditions include, but are not limited to, female infertility and ovarian cancer. Other diseases and conditions associated with the development and function of the ovaries are encompassed within the scope of this invention.

Since LGR6-SVs polypeptide is likely to play a role in cell proliferation and differentiation, LGR6-SVs nucleic acid molecules, polypeptides, and agonists and antagonists thereof may be useful in diagnosing or treating diseases and conditions that modulate cell proliferation and differentiation. For example, the LGR6-SVs molecules of the invention may be used to increase cell proliferation and differentiation. Examples of diseases and conditions that may be treated by increasing cell proliferation and differentiation include, but are not limited to, tissue damage and degeneration (such as that caused by cancer therapy, infections, autoimmune diseases, or disorders), aging, and wound healing. Other diseases and conditions that could be treated by increasing cell proliferation and differentiation are encompassed within the scope of this invention.

Alternatively, the LGR6-SVs molecules of the invention may be used to decrease cell proliferation and differentiation. Examples of diseases and conditions that may be treated by decreasing cell proliferation and differentiation include, but are not limited to, cancer, hyperplasia, and hypertrophy. Other diseases and conditions that could be treated by decreasing cell proliferation and differentiation are encompassed within the scope of this invention.

Agonists or antagonists of LGR6-SVs polypeptide function may be used (simultaneously or sequentially) in combination with one or more cytokines, growth factors, antibiotics, anti-Inflammatories, and/or chemotherapeutic agents as is appropriate for the condition being treated.

Other diseases or disorders caused by or mediated by undesirable levels of LGR6-SVs polypeptides are encompassed within the scope of the invention. Undesirable levels include excessive levels of LGR6-SVs polypeptides and sub-normal levels of LGR6-SVs polypeptides.

Uses of LGR6-SVs Nucleic Acids and Polypeptides

Nucleic acid molecules of the invention (including those that do not themselves encode biologically active polypeptides) may be used to map the locations of the LGR6-SVs gene and related genes on chromosomes. Mapping may be done by techniques known in the art, such as PCR amplification and in situ hybridization.

LGR6-SVs nucleic acid molecules (including those that do not themselves encode biologically active polypeptides), may be useful as hybridization probes in diagnostic assays to test, either qualitatively or quantitatively, for the presence of an LGR6-SVs nucleic acid molecule in mammalian tissue or bodily fluid samples.

Other methods may also be employed where it is desirable to inhibit the activity of one or more LGR6-SVs polypeptides. Such inhibition may be effected by nucleic acid molecules that are complementary to and hybridize to expression control sequences (triple helix formation) or to LGR6-SVs mRNA. For example, antisense DNA or RNA molecules, which have a sequence that is complementary to at least a portion of an LGR6-SVs gene can be introduced into the cell. Anti-sense probes may be designed is by available techniques using the sequence of the LGR6-SVs gene disclosed herein. Typically, each such antisense molecule will be complementary to the start site (5 end) of each selected LGR6-SVs gene. When the antisense molecule then hybridizes to the corresponding LGR6-SVs mRNA, translation of this mRNA is prevented or reduced. Anti-sense inhibitors provide information relating to the decrease or absence of an LGR6-SVs polypeptide in a cell or organism.

Alternatively, gene therapy may be employed to create a dominant-negative inhibitor of one or more LGR6-SVs polypeptides. In this situation, the DNA encoding a mutant polypeptide of each selected LGR6-SVs polypeptide can be prepared and introduced into the cells of a patient using either viral or non-viral methods as described herein. Each such mutant is typically designed to compete with endogenous polypeptide in its biological role.

In addition, an LGR6-SVs polypeptide, whether biologically active or not, may be used as an immunogen, that is, the polypeptide contains at least one epitope to which antibodies may be raised. Selective binding agents that bind to an LGR6-SVs polypeptide (as described herein) may be used for in vivo and in vitro diagnostic purposes, including, but not limited to, use in labeled form to detect the presence of LGR6-SVs polypeptide in a body fluid or cell sample. The antibodies may also be used to prevent, treat, or diagnose a number of diseases and disorders, including those recited herein. The antibodies may bind to an LGR6-SVs polypeptide so as to diminish or block at least one activity characteristic of an LGR6-SVs polypeptide, or may bind to a polypeptide to increase at least one activity characteristic of an LGR6-SVs polypeptide (including by increasing the pharmacokinetics of the LGR6-SVs polypeptide).

LGR6-SVs polypeptides can be used to clone LGR6-SVs ligands using an “expression cloning strategy. Radiolabeled (125 Iodine) LGR6-SVs polypeptide or “affinity/activity tagged” LGR6-SVs polypeptide (such as an Fc fusion or an alkaline phosphatase fusion) can be used in binding assays to identify a cell type, cell line, or tissue that expresses an LGR6-SVs ligand. RNA isolated from such cells or tissues can then be converted to cDNA, cloned into a mammalian expression vector, and transfected into mammalian cells (e.g., COS or 293) to create an expression library. Radiolabeled or tagged LGR6-SVs polypeptide can then be used as an affinity reagent to identify and isolate the subset of cells in this library expressing an LGR6-SVs ligand. DNA is then isolated from these cells and transfected into mammalian cells to create a secondary expression library in which the fraction of cells expressing the LGR6-SVs ligand would be many-fold higher is than in the original library. This enrichment process can be repeated iteratively until a single recombinant clone containing the LGR6-SVs ligand is isolated. Isolation of LGR6-SVs ligands is useful for identifying or developing novel agonists and antagonists of the LGR6-SVs signaling pathway. Such agonists and antagonists include LGR6-SVs ligands, antiLGR6-SVs ligand antibodies, small molecules or antisense oligonucleotides.

The murine and human LGR6-SVs nucleic acids of the present invention are also useful tools for isolating the corresponding chromosomal LGR6-SVs polypeptide genes. For example, mouse chromosomal DNA containing LGR6-SVs sequences can be used to construct knockout mice, thereby permitting an examination of the in vivo role for LGR6-SVs polypeptide. The human LGR6-SVs genomic DNA can be used to identify heritable tissue-degenerating diseases.

The following examples are intended for illustration purposes only, and should not be construed as limiting the scope of the invention in any way.

### EXAMPLES

### Example 1

### Identification of LGR6.1 and LGR6.2

The NCBI Data Bases were searched using human LGR6 nucleotide sequence as query sequence. This search identified a novel variant of the LGR6 receptor. This variant was named LGR6.1. LGR6.1 differs from LGR6 both at the N-terminal as well as C-terminal region. LGR6.1 encodes 3 extra Leucine rich repeats and a distinct leader peptide. The C-terminus of LGR6.1 differs from the published LGR6 sequence. The novel C-terminus of LGR6.1 is F-A-S-H-V and interestingly this is a perfect PDZ binding motif for class I consensus PDZ domain (X-S/T-X-V).

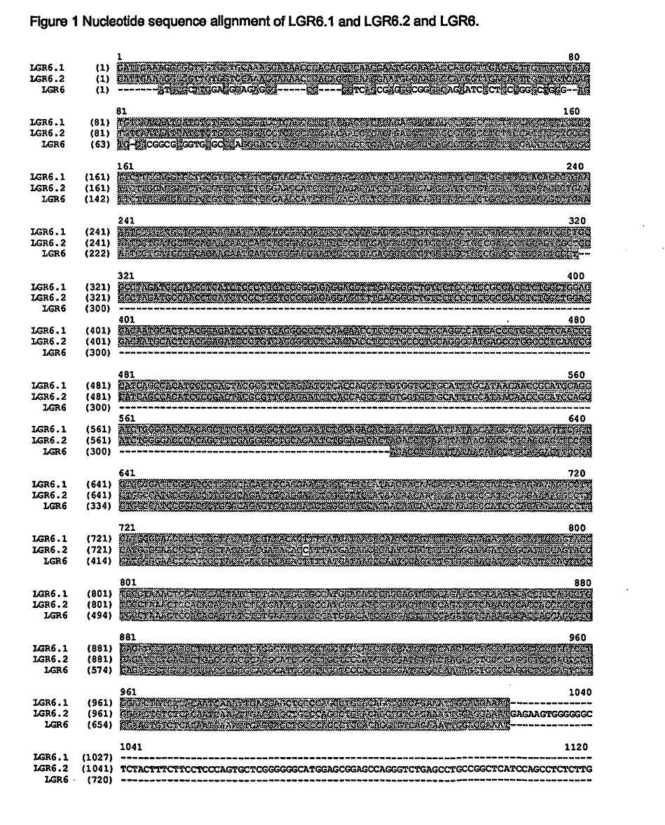

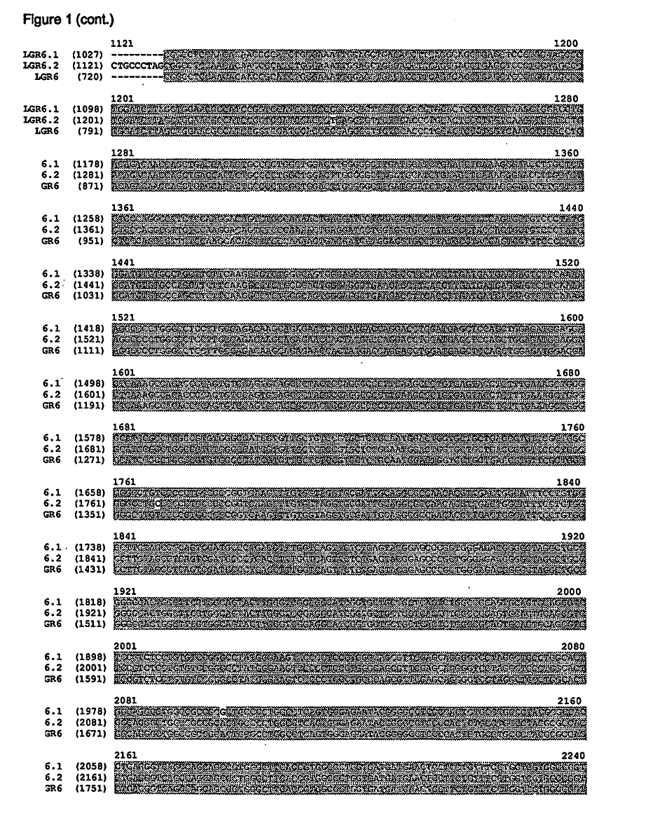

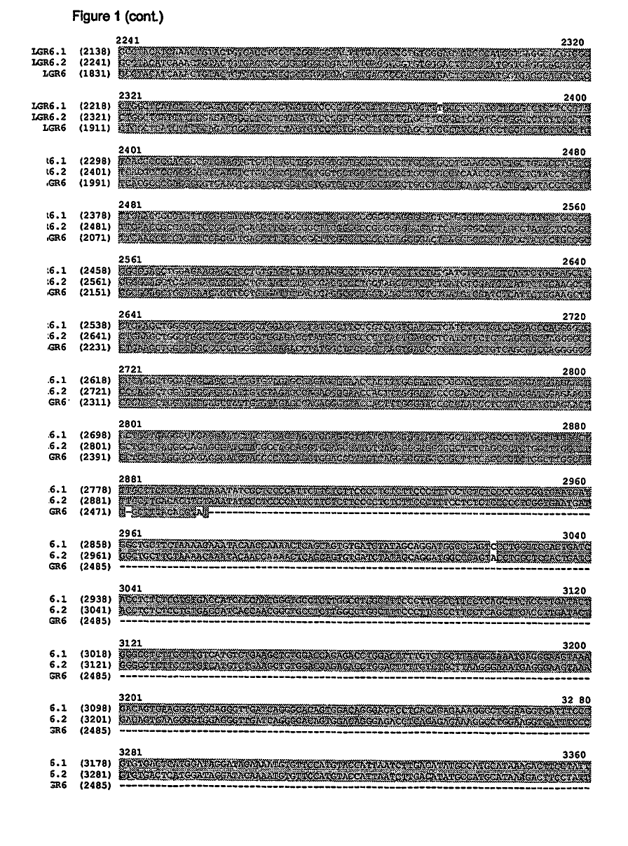

Furthermore, we have obtained the EST done corresponding to dbEST Id: 9815955 (partially matching with the query sequence) from Research Genetics and completed the sequence. We have in this way identified a shorter, possibly soluble, splice variant of LGR6.1 receptor. We have named it LGR6.2. Interestingly, LGR6.2 encodes a novel exon that contains a novel stop codon resulting in a protein of 348 amino acids. Similar to a short version of the TSH receptor, LGR6.2 contains only the leucine rich repeat extracellular portion. Because LGR6.2 does not contain the seven-transmembrane domain it could represent a novel secreted protein, which may act as LGR6.1 antagonist in vivo. The alignment of the nucleotide sequences coding for LGR6.1, LGR6.2 and LGR6 is reported in FIG. 1.

To determine the signal peptide of these 2 new spliced variants SignalP™ predictions using neural networks (NN) and hidden Markov (HMM) models were rum on the sequences. Both predictive models resulted in an approximate 50% probability that the first 19 amino acids represent the signal peptide.

### Example 2

### Tissue Distribution of LGR6.1 and LGR6.2 in Human Tissue

TaqMan Analysis for LGR6.1 and LGR6.2

1 μg total RNA from 21 different human tissues (0.5 μg PolyA+RNA from human pituitary gland) were reverse-transcribed by MMLV-reverse transcriptase.

2.5 μl of the RT reaction were tested by TaqMan analysis according to the manufacturer protocol (1×TaqMan universal mix, 900 nM forward and reverse primers and 250 nM probe in 25 id final volume). No template control was run as a negative control, as well as samples minus Reverse Transcriptase enzyme. A standard curve was run using plasmids encoding LGR6.1 and LGR6.2 to calculate copy number. β-actin was chosen as internal control (Applied Biosystem).

Taqman was performed on a ABIPRISM 7700 Sequence Detector (Applied Biosystems) and the conditions were as follow: 1 cycle 50° C. 2 min., 1 cycle 95° C. 10 min., 40 cycles 95° C. 15 sec. and 60° C. 1 min.

The following primers have been used:

| | |
|---|---|
| LGR6.2 fwd: | ATTGGAGGAAATGAGAAGTGGG |
| LGR6.2 rev: | CCGCTAGGGCAGCAAGAG |
| LGR6.2 probe: | 6FAM-CATGGAGCGGAGCCAGGGTCTG-TAMRA |
| LGR6.1 240 fwd: | AAATCCTGATGCTGCAGAACAA |
| LGR6.1 358 rev: | CCTCTCCGGGACCAGGG |
| LGR6.1 probe: | 6FAM-CTGCCGAGCCTGCAGTCGCTG-TAMRA |

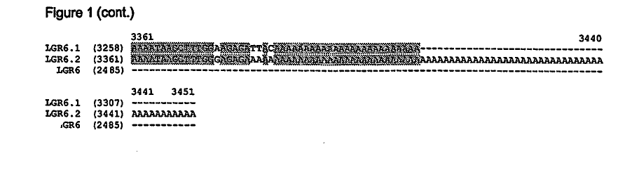

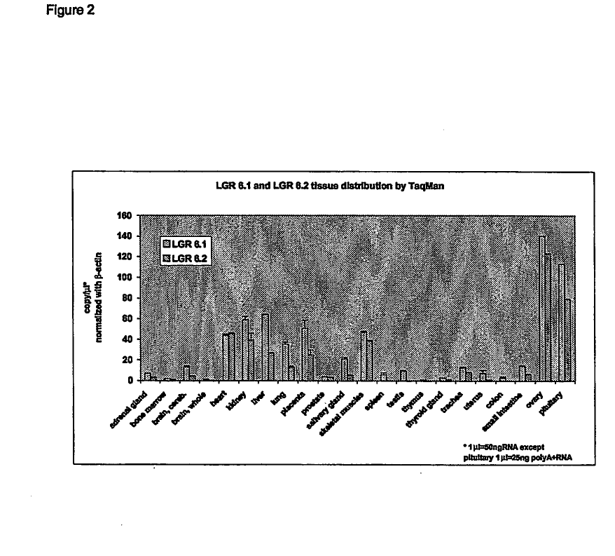

A picture showing the tissue expression distribution of the 2 novel sequences is shown in FIG. 2.

### Example 3

### Identification and Description of LGR6 Domains

3.1 Identification

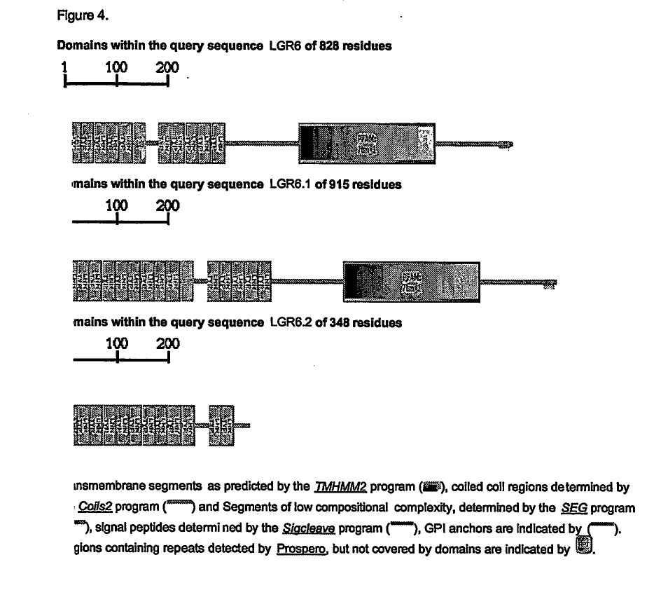

A bioinformatic tool called SMART (http://smart.embl-heidelberg.de/) was used to identify the putative domains of the splice variants LGR6.1 and LGR6.2. Results are shown in FIG. 4. In addition, Prosite was also run on the sequences (http://us.expasy.org/prosite/.

Partial ScanProsite Results:

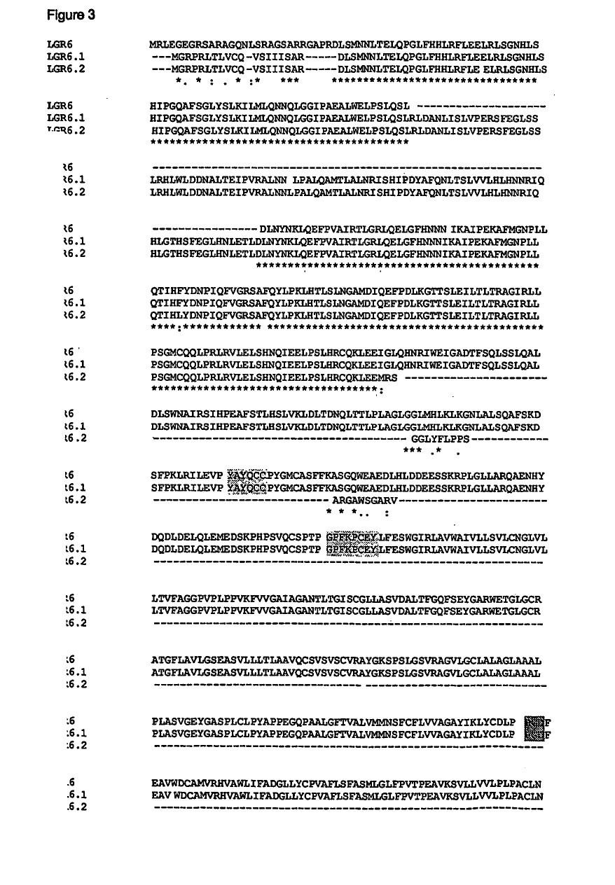

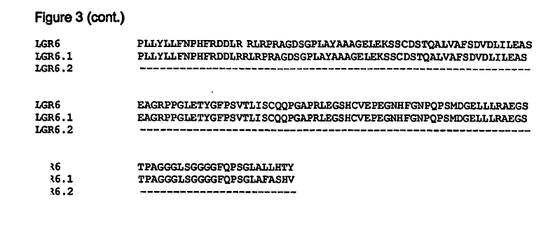

LGR6:
>PDOC00016 PS00016 RGD Cell attachment sequence [pattern] [Warning: pattern with a high probability of occurrence]. See FIG. 3.
   621-623 RGD

LGR6.1:
>PDOC00016 PS00016 RGD Cell attachment sequence [pattern] [Warning: pattern with a high probability of occurrence]. See FIG. 3.
   708-710 RGD
In addition, conserved amino acid sequences in the hinge regions (located between the leucine-rich repeats and the transmembrane domain) were identified. LGR4, LGR5 and LGR6 can be subdivised in a particular LGR subgroup because they share in common the same hinge conserved sequences corresponding to YAYQCC and GXFKPCEX LGR6.1 but not LGR6.2 contain these conserved sequences (shown in FIG. 3)
   3.2 Description of the Domains

LRRs
Leucine-rich repeats (LRRs) are relatively short motifs (22-28 residues in length) found in a variety of cytoplasmic, membrane and extracellular proteins. Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined revealing LRRs to be a new class of alpha/beta fold. LRRs form elongated non-globular structures and are often flanked by cysteine rich domains.

7 Transmembrane Receptor (Rhodopsin Family)
G-protein-coupled receptors, GPCRs, constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence MEDLINE:94224751. The currently known clan members include the rhodopsin-like GPCRs, the secretin-like GPCRs, the CAMP receptors, the fungal mating pheromone receptors, and the metabotropic glutamate receptor family. There is a specialized database for GPCRs: http://www.gpcr.org/7tm/. The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices MEDLINE:90262152. MEDLINE:88139292, MEDLINE:93234436.

PS00016; RGD
The sequence Arg-Gly-Asp, found in fibronectin, is crucial for its interaction with its cell surface receptor, an integrin. What has been called the ‘RGD’ tripeptide is also found in the sequences of a number of other proteins, where it has been shown to play a role in cell adhesion. These proteins are: some forms of collagens, fibrinogen, vitronectin, von Willebrand factor (VWF), snake disintegrins, and slime mold discoidins. The ‘RGD’ tripeptide is also found in other proteins where it may also, but not always, serve the same purpose.
   3.3 Conclusion
In addition to the comments put forward in example 1, it is clear, based on SMART results (FIG. 4), that LGR6.1 is closely related to LGR6 and that LGR6.2 indeed represents a novel secreted protein. Interestingly, LGR6 and LGR6.1 contain an RGD tripeptide sequence. Indicating that these proteins may interact with integrin cell surface receptors, which are involved in cell adhesion. LGR6.2 is lacking the tripeptide sequence, which is in support of LGR6.2 as being a soluble protein.
Furthermore, LGR6.1 but not LGR6.2 possess the hinge-conserved sequences of the LGR subgroup composed of LGR4, LGR5 and LGR6. As such a particular activation mechanism seems to be involved for this particular LGR subgroup. Yu Hsu et al. suggest that these receptors may share similar ligand binding and signal transduction characteristics (Sheau Yu Hsu et al. 2000. Molecular Endocrinology 14(8):1257-1271). They also point out, as this particular LGR subgroup share similar structural determinants in the hinge region, that investigations on this region of the orphan receptors could provide insights toward the activation mechanisms of different LGRs. Furthermore, they state that recent studies on the TSH receptor have shown that point mutation of the serine residue in the conserved YPSHCC motif resulted in constitutive activation of the TSH receptor, leading to severe congenital hyperthyroidism. In a similar way, critical mutations in the hinge-conserved sequences of LGR6 or LGR6.1 could constitutively activate these receptors, leading to particular congenital diseases or cancers. The secreted LGR6.2 might be able to counteract some forms of constitutive or excess activation of the LGR subgroup composed of LGR4, LGR5 and LGR6.

### Example 4

### Reproductive Health Assays Suitable for Exploration of the Biological Relevance of Proteins Function

A number of reproductive health-related assays have been developed by the Applicant and are of use in the investigation of the biological relevance of LGR6 protein function. In view of the high expression of LGR6.1 and LGR6.2 in the ovaries (example 2), such assays seem of particular relevance. Examples of reproductive health-related assays that have been developed by the Applicant include 18 cell-based assays for reproductive health. These are discussed below.

4.1 Primary Human Uterine Smooth Muscle Proliferation Assay:
The proliferation of uterine smooth muscle cells is a precursor for development of tumors in uterine fibroid disease in women. In this assay, the goal is to identify proteins that inhibit proliferation of primary human uterine smooth muscle cells.

4.2 JEG-3 Implantation Assay:
JEG-3 cells are a choriotrophoblastic human cancer cell line used as a model for the blastocyst during implantation. Ishikawa cells are a relatively non-differentiated endometrial human cancer cell line that is used as a model for the decidua. JEG-3 cells will “implant” into human decidual tissue. In this assay, a 2-chamber system is used where fluorescently labeled JEG-3 cells invade through a Matrigel-coated porous membrane from an upper chamber into a lower chamber when Ishikawa cells or Ishikawa-conditioned medium are placed into the lower chamber. The cells that migrate are quantified in a plate reader. The goal is to identify proteins that increase invasion of JEG-3 cells for use in aiding implantation in vivo.

4.3 Osteopontin Bead Assay (Ishikawa Cells):
Ishikawa human endometrial cancer cells are used as a model for implantation. At the time of implantation in the human, various integrins are expressed by the uterine endometrium that is thought to bind to proteins expressed by the blastocyst. Ishikawa cells have been shown in the literature to express avb3, which is the integrin expressed by the uterine endometrium during the “window of implantation”. This integrin is believed to bind the osteopontin expressed by the trophoblast. In this assay, osteopontin-coated fluorescent beads represent the blastocyst, and the Ishikawa cells are primed to accept them for binding by treating them with estradiol. The goal is to identify proteins that increase the ability of the Ishikawa cells to bind the osteopontin-beads as an aid to increase receptivity of the uterine endometrium at the time of implantation.

4.4 HuF6 Assay:
HuF6 cells are primary human uterine fibroblast cells. These cells can be induced to decidualize by treating them with IL-1β. A marker for decidualization is production of PGE2, which is measured by ELISA. The goal is to identify proteins that increase production of PGE2 by the HuF6 cells as a way of enhancing decidualization during early pregnancy.

4.5 Endometriosis Assay:
Peritoneal TNFα plays a role in endometriosis by inducing the sloughed endometrial cells from the uterus to adhere to and proliferate on peritoneal mesothelial cells. In this assay, BEND cells are treated with TNFα, which increases their ability to bind fibronectin-coated fluorescent beads as an assay for adherence during endometriosis. The goal is to identify proteins that decrease or inhibit the ability of TNFα to stimulate bead-binding capacity of the cells.

4.6 Cyclic AMP Assay Using JC-410 Porcine Granulose Cells Stably Transfected with hLHR:
In Polycystic Ovary Syndrome, LH from the pituitary is relatively high, and induces androgen output from the ovarian thecal cells. This assay is used to look for an inhibitor of LH signaling which could be used to decrease the action of LH at the ovary during PCOS. The JC-410 porcine granulosa cell line is stably transfected with the human LH receptor. Treatment with LH results in CAMP production.

4.7 Cyclic AMP Assay Using JC-410 Porcine Granulose Cells Stably Transfected with hFSHR:
The JC-410 porcine granulosa cell line was stably transfected with the human FSHR. Treatment with FSH stimulates CAMP production, which is measured in this assay. The goal is to identify proteins that enhance FSH action in the granulosa cells.

4.8 LbetaT2 (Mouse) Pituitary Cells Assay:
The LbetaT2 is an immortalized murine pituitary gonadotroph cell line. Stimulation with Activin alone or with GnRH+Activin results in secretion of FSH (stimulation with GnRH alone results in secretion of LH.) The cells can either be treated with GnRH+Bioscreen proteins to find proteins that act in concert with GnRH to stimulate FSH production, or they can be treated with Bioscreen proteins alone to find a protein that can stimulate FSH secretion like activin alone.

4.9 Cumulus expansion assay:
The cumulus-expansion assay using murine cumulus-oocyte complexes (2/well) has been validated in a 96-well format to assay for proteins that affect oocyte maturation (measured by cumulus expansion). Two 96-well plates can be processed per assay, and 2 assays per week can be performed. If Bioscreen proteins are assayed at only one concentration, all Bioscreen I proteins can be assayed in a month. The read-out may be a yes/no answer for expansion, or image analysis programs may be used to measure expansion in a quantitative manner.

4.10 RWPE Proliferation Assay:
Benign prostatic hyperplasia is characterized by growth of prostatic epithelium and stroma that is not balanced by apoptosis, resulting in enlargement of the organ. RWPE is a regular human prostatic epithelial cell line that was immortalized with the HPV-18, and may be used in place of primary human prostatic epithelial cells.

4.11 HT-1080 Fibrosarcoma Invasion Assay:
This assay was developed as a positive cell control for the JEG-3 implantation assay (above). This is a well-established assay as a model for cancer metastasis. Fluorescently-labeled HT-1080 human fibrosarcoma cells are cultured in the upper chamber of a 2-chamber system, and can be stimulated to invade through the porous Matrigel-coated membrane into the bottom chamber where they are quantified. The goal is to identify a protein that inhibits the invasion. The cells are stimulated to invade by adding serum to the bottom chamber and are inhibited with doxycycline.

4.12 Primary Human Uterine Smooth Muscle Assay:
One of the hallmarks of uterine fibroid disease is collagen deposition by the uterine smooth muscle cells that have become leioymyomas. Primary human uterine smooth muscle cells are stimulated to produce collagen by treatment with TGFβ, which is blocked with Rebif. The goal is to discover proteins that inhibit this fibrotic phenotype.

4.13 Human Leiomyoma Cells Proliferation Assay:
A human leiomyoma cell line may be used as a model for uterine fibroid disease in a proliferation assay. The cells grow very slowly and we are stimulating them to grow at a faster rate by treating them with estradiol and growth factors. The goal is to identify proteins that inhibit estradiol-dependent growth of leiomyoma cells.

4.14 937 Migration Assay:
Endometriotic lesions secrete cytokines that recruit immune cells to the peritoneal cavity. These immune cells (especially activated macrophages and T lymphocytes) mediate inflammatory symptoms that are common to endometriosis. RANTES has been shown to be produced by endometriotic stromal cells and is present in the peritoneal fluid. In this assay, U937, a monocytic cell line used as a model for activated macrophages, can be induced by treating the lower level of a 2-chamber culture system to migrate from the upper chamber. If the cells are pre-loaded with fluorescent dye, they can be quantified in the lower chamber. The goal is to identify proteins that inhibit the migration of the U937 cells.

4.15 JEG3 Human Trophoblast Assay:
The trophoblast of the blastocyst produces HLA-G, a class I HLA molecule that is believed to be important in preventing immunological rejection of the embryo by the mother. During pre-eclampsia, HLA-G levels are low or non-existent, presumably resulting in hallmark symptoms such as poor invasion of the trophoblast into the endometrium and spiral arteries because of maternal immunological interference. The JEG-3 human trophoblast cell line produces HLA-G, which can be increased by treatment with IL-10 or LIF. An ELISA can be used to measure HLA-G production by JEG3 cells, with the goal being the discovery of other proteins that can increase HLA-G production.

4.16 Primary Rat Ovarian Dispersate Assay:
Due to the difficulties in measuring appreciable amounts of steroids from the JC-410-FSHR/LHR cell lines, an assay using primary cells from whole ovaries taken from immature rats has been developed. Initially, estradiol production from these cultures is measured after treatment with FSH and/or LH. The goal is then to identify proteins that enhance gonadotropin-stimulated steroidogenesis, or proteins that work alone to increase steroidogenesis by these cultures.

4.17 Mouse IVF Assay:
In this assay, sperm function, measured by ability to fertilize oocytes, is assayed with the goal of finding proteins that stimulate fertilizing potential of sperm.

4.18 Primary Human Prostate Stromal Cells Proliferation Assay:
An assay for the epithelial component of BPH has already been described above (see RWPE assay above). This assay uses primary human prostate stromal cells as a model for proliferation of these cells during BPH. The goal is to identify proteins that inhibit proliferation of these cells.

While the present invention has been described in terms of the preferred embodiments, it is understood that variations and modifications will occur to those skilled in the art. Therefore, it is intended that the appended claims cover all such equivalent variations that come within the scope of the invention as claimed.

1. An isolated nucleic acid molecule comprising a nucleotide sequence selected from the group consisting of:
(a) the nucleotide sequence as set forth in any of SEQ ID NO: 1 or SEQ ID NO: 3;
(b) a nucleotide sequence encoding a polypeptide at least 85% identical to one of:
the polypeptide as set forth in SEQ ID NO: 2, wherein the encoded polypeptide has an activity of the polypeptide set forth in SEQ ID NO: 2; or
the polypeptide as set forth in SEQ ID NO: 4, wherein the encoded polypeptide has an activity of the polypeptide set forth in SEQ ID NO: 4; and
(c) a nucleotide sequence which hybridizes under moderately stringent conditions with one of:
(a) or (b); or
the nucleotide sequence 1-102 of SEQ ID NO: 1 or 1-102 of SEQ ID NO:3; or
the nucleotide sequence 319-606 of SEQ ID NO:1 or 319-606 of SEQ ID NO: 3; or
the nucleotide sequence 1027-1201 of SEQ ID NO: 3; or
a nucleotide sequence encoding a polypeptide as set forth in any of SEQ ID NO: 2 or SEQ ID NO: 4, with one conservative amino acid substitution, wherein the encoded polypeptide has an activity of the polypeptide set forth in any of SEQ ID NO: 2 or SEQ ID NO: 4.

2.-3. (canceled)

4. A vector comprising the nucleic acid molecule of claim 1 and a promoter operatively linked to the nucleic acid molecule.

5. A host cell comprising the vector of claim 4.

6. The host cell of claim 5 that is a eukaryotic cell.

7. The host cell of claim 5 that is a prokaryotic cell.

8. A process of producing an LGR6-SVs polypeptide comprising culturing the host cell of claim 5 under suitable conditions to express the polypeptide, and optionally isolating the polypeptide from the culture.

9. A polypeptide encoded by the nucleotide sequences of claim 1.

10. The process of claim 8, wherein the promoter is not the native promoter for the LGR6-SVs polypeptide.

11. (canceled)

12. A process for determining whether a compound inhibits LGR6-SVs polypeptide activity or LGR6-SVs polypeptide production comprising:
(a) exposing a cell according to claim 5 to the compound; and
(b) measuring one of:
LGR6-SVs polypeptide activity in said cell, wherein a decrease in activity indicates that the compound inhibits LGR6-SVs polypeptide activity; or
LGR6-SVs polypeptide production in said cell, wherein a decrease in production indicates that the compound inhibits LGR6-SVs polypeptide production.

13. The polypeptide of claim 9 wherein the polypeptide is characterized by the following:
(a) the polypeptide comprises the sequence set forth in SEQ ID NO: 2;
(b) the polypeptide comprises the sequence set forth in SEQ ID NO: 4;
(c) the polypeptide comprises the sequence set forth in SEQ ID NO: 2 with one conservative amino acid substitution, wherein the polypeptide has an activity of the polypeptide set forth in SEQ ID NO: 2;
(d) the polypeptide comprises the sequence set forth in SEQ ID NO: 4 with one conservative amino acid substitution, wherein the polypeptide has an activity of the polypeptide set forth in SEQ ID NO: 4.

14. The polypeptide according to claim 13, wherein the polypeptide does not comprise a signal sequence.

15. An antibody that specifically binds the polypeptide of claim 13.

16. The antibody of claim 15 wherein the antibody specifically binds a polypeptide comprising the amino acid sequence as set forth in any of SEQ ID NO: 2 or SEQ ID NO: 4.

17. The antibody of claim 16 wherein the antibody comprises a Fab or F(ab′) fragment.

18. The antibody of claim 17 wherein the antibody is a humanized antibody.

19. A method for treating, preventing, or ameliorating an LGR6-SVs polypeptide-related disease, condition, or disorder comprising administering to a patient an antibody according to claim 16.

20.-22. (canceled)

23. A composition comprising the polypeptide of claim 13 and a pharmaceutically acceptable formulation agent.

24.-26. (canceled)

27. The polypeptide of claim 13 wherein the polypeptide is covalently modified with a water-soluble polymer selected from the group consisting of polyethylene glycol, mono-methoxy polyethylene glycol, dextran, cellulose, poly-(n-vinyl pyrrolidone) polyethylene glycol, propylene glycol homopolymers, polypropylene oxide/ethylene oxide copolymers, polyoxyethylated polyols, and polyvinyl alcohol.

28.-31. (canceled)

32. The polypeptide of claim 13 wherein the polypeptide is a fusion protein.

33. The fusion polypeptide of claim 32, wherein the fusion protein comprises an IgG constant domain or fragment thereof.

34. (canceled)
